(19) United States
(12) Patent Application Publication
(10) Pub. No.: US 2025/0356959 A1
(43) Pub. Date: Nov. 20, 2025

(54) Toxicity Prediction Of Compounds In Cellular Structures

(71) Applicant: Sanofi, PARIS (FR)

(72) Inventors: Reza Olfati-Saber, Cambridge, MA (US); Elton Rexhepaj, Paris (FR); Rachid Boukaiba, Paris (FR); Pascale Roux, Paris (FR); Michel Partiseti, Paris (FR)

(21) Appl. No.: 19/120,392

(22) PCT Filed: Oct. 13, 2023
(86) PCT No.: PCT/EP2023/078554

§ 371 Date: Apr. 11, 2025

(30) Foreign Application Priority Data
Oct. 17, 2022 (EP) 22290056.5

(51) Int. Cl. G16C 20/30; G06T 7/00; G16B 15/00; G16H 30/20

(52) U.S. Cl. CPC G16C 20/30; G06T 7/0014; G16B 15/00; G16H 30/20; G06T 2207/20081; G06T 2207/20084; G06T 2207/30024

(57) ABSTRACT

Methods, apparatus and systems are disclosed for predicting toxicity of one or more compounds applied to a plurality of samples of a cellular structure in an in-vitro microscopy assay. A set of images associated with the plurality of samples are received. Each image of the set of images are input to a first ML model configured for predicting phenotype features of the cellular structure within the sample associated with said each image. Each of the predicted phenotype features associated with each sample are input to a second ML model configured for predicting a lower dimensional phenotype feature embedding of said each sample. The distance between the lower dimensional phenotype feature embedding of said each sample is compared with that of a sample applied with a compound having a known toxicity. For each sample, an indication of the toxicity of said each sample and applied compound thereto is output based on said comparison.

### CROSS REFERENCE TO RELATED APPLICATIONS

The present application is the national stage entry of International Patent Application No. PCT/EP2023/078554, filed on Oct. 13, 2023, and claims priority to Application No. EP 22290056.5, filed on Oct. 17, 2022, the disclosures of which are incorporated herein by reference.

### TECHNICAL FIELD

This specification relates to apparatus, systems and method(s) for predicting the toxicity of compounds in cellular structures of microscopy assay samples.

### BACKGROUND

Cellular structures have been developed that may mimic and/or simulate the processes and/or functions of an organ of a subject or patient. These can be used for in-vitro testing of the efficacy and/or toxicity of various compounds in relation to an organ instead of in-vivo testing. Such cellular structures include immortalised cell-lines that have been developed to mimic or simulate a particular organ of a subject. This has led to semi-automated toxicity prediction test systems and methodologies that may be used to identify compounds that are toxic to an organ of a subject using image microscopy and observing changes due to toxicity with dose-response (DR) graphs and the like.

### SUMMARY

Conventional semi-automated test systems and methodologies can be used to identify compounds that produce a detectable signal to assess the effect that compounds have on the cellular structures in relation to an organ. These test systems are called assays. Once an assay has been developed for toxicity prediction, researchers can use it to identify compounds that have the required activity in relation to toxicity. Typically, a compound will be tested at a number of concentrations, imaged using image microscopy and a DR graph or other metric may be generated that is useful for researchers to determine its toxicity. For example, analysis of the DR graph may allow researchers to determine if a compound is active and/or toxic, and at what concentration.

It is desirable to test a large number of potential compounds in which High Throughput Screening (HTS) is often used. This uses robotics, data processing/control and imaging software, liquid handling devices and sensitive detectors, and allows researchers to quickly conduct thousands or even millions of screening tests. However, the large amount of data generated at the imaging and DR steps of a HTS campaign requires careful analysis by researchers in order to detect artifacts and correct erroneous data points before validating the experiments.

Unfortunately, even semi-automated toxicity prediction assays using HTS have been found to be unable to reliably identify the toxicity of every compound, even compounds known to be toxic when analysed on cellular structures simulating/mimicking an organ. This has increased the risks of performing in-vivo trials of compounds that have passed such semi-automated toxicity prediction assays. For example, 20-40% of drug-induced liver injury (DILI) patients present a cholestatic and/or mixed hepatocellular/cholestatic injury pattern. Drug-induced hepatotoxicity or DILI is an acute or chronic response to a natural or manufactured compound. Conventionally, DILI can be classified based on clinical presentation (hepatocellular, cholestatic, or mixed), mechanism of hepatotoxicity, or histological appearance from a liver biopsy. Thus, reliable in-vitro toxicity prediction of compounds is an important component of a drug/compound discovery or research programme.

There is a desire for an improved methodology, apparatus, systems and/or an architecture capable of efficiently and reliably detecting or predicting the toxicity of compounds on cellular structures using in-vitro HTS assays and the like.

According to a first aspect, there is provided a computer-implemented method for predicting toxicity of one or more compounds applied to a plurality of samples of a cellular structure in an in-vitro microscopy assay, the method comprising: receiving a set of images associated with the plurality of samples; inputting each image of the set of images to a first ML model configured for predicting phenotype features of the cellular structure within the sample associated with said each image; inputting each of the predicted phenotype features associated with each sample to a second ML model configured for predicting a lower dimensional phenotype feature embedding of said each sample; comparing the distance between the lower dimensional phenotype feature embedding of said each sample with that of a sample applied with a compound having a known toxicity; and outputting, for each sample, an indication of the toxicity of said each sample and applied compound thereto based on said comparison.

The computer-implemented method of the first aspect, wherein the first ML model is a neural network or convolutional neural network (CNN) model. As an option, the neural network or CNN model is trained for classification using cellular image training data. As another option, the predicted phenotype features are embedded within a full layer of the trained neural network or CNN model, the method comprising outputting the phenotype features from the full layer. Optionally, the final full layer of the neural network or CNN model is used to output an embedding of said phenotype features.

The computer-implemented method of the first aspect, wherein the second ML model is based on a Uniform Manifold Approximation and Projection (UMAP) algorithm or t-SNE algorithm for dimensional reduction of the phenotype feature embedding of a sample, wherein the phenotype feature embedding is mapped to a lower dimensional vector space for use in comparing the distance between the phenotype feature embedding and that of a sample with a compound having a known toxicity.

As an option, the second ML model is trained on the UMAP technique using unsupervised training based on negative and positive control samples of the plurality of samples for predicting a toxicity distance metric associated with the samples with compounds applied thereto having a known toxicity. As another option, training the second ML model comprises iteratively performing a grid search over a set of hyperparameters of the UMAP technique for selecting those hyperparameters that maximise the differences between the negative control samples and the positive control samples.

The computer-implemented method of the first aspect, wherein indicating the toxicity of the phenotype feature embedding of a sample with compound applied thereto comprises applying the phenotype feature embedding of a sample with compound applied thereto to the second ML model for outputting a lower dimensional embedding of said sample with compound applied thereto; and determining an indication of the toxicity of said sample with compound applied thereto based on comparing the distance between said lower dimensional embedding and the embeddings of one or more samples with compounds applied thereto having known toxicity.

The computer-implemented method of the first aspect, wherein indicating the toxicity of the phenotype feature embedding of a sample with compound applied thereto further comprising: applying the phenotype feature embedding of a sample with compound applied thereto to the second ML model for outputting a lower dimensional embedding of said sample with compound applied thereto; and applying the lower dimensional embedding of said sample with compound applied thereto to a third ML model trained for outputting an indication of the distance between the lower dimensional embedding and a set of the lower dimensional embeddings associated with the negative control samples.

The computer-implemented method of the first aspect, further comprising training the third ML model based on performing a grid search over a set of hyperparameters of a high dimensional distance metric algorithm that maximise a distance between the lower dimensional embeddings of the negative control samples and the positive control samples, whilst minimising the distance between the lower dimensional embeddings of the negative control samples or minimising the distance between the lower dimensional embeddings of the positive control samples.

The computer-implemented method of the first aspect, wherein the distance metric is the Wasserstein distance metric and the high dimensional distance metric algorithm is the Sinkhorn algorithm for estimating the Wasserstein distance between embeddings.

The computer-implemented method of the first aspect, wherein comparing the distance used for indicating the toxicity of the phenotype feature embedding of a sample is based on Wasserstein distance metrics.

The computer-implemented method of the first aspect, wherein receiving said set of images associated with the plurality of samples further comprising: identifying viable samples of cellular structures for analysis in an in-vitro microscopy assay, based on the steps of: automatically identifying a first set of samples useful for analysis from a plurality of samples of an assay plate; generating a set of 2-dimensional (2D) images for each sample in the first set of samples, said set of 2D images for said each sample comprising multiple 2D image slices taken along a z-axis of said each sample; and identifying from the sets of 2D image slices a set of viable samples; and outputting data representative of said set of viable samples for analysis as the set of images.

The computer-implemented method of the first aspect, wherein automatically identifying a first set of samples further comprising, for each sample in the plurality of samples: pre-processing an image of said each sample; inputting said pre-processed sample image to a first machine learning, ML, model configured for identifying a region of interest of the input sample image comprising a cellular structure; inputting the identified region of interest of sample image to a second ML model configured for classifying whether said sample is analysable; and outputting the first set of samples comprising data representative of those samples that are classified to be analysable.

The computer-implemented method of the first aspect, wherein: the first ML model is a convolutional neural network, CNN, or other neural network trained for identifying regions of interest comprising cellular structures, and the second ML model is a one class SVM configured to classify whether said region of interest is analysable.

The computer-implemented method of the first aspect, wherein: training/configuring the CNN based on a labelled training dataset, said labelled training dataset comprising a plurality of images, each of the images annotated with a label comprising data representative of whether a cellular region of interest is present, and/or the location of the region of interest within the image etc.

As an option, training/configuring the one class SVM to classify whether said region of interest is analysable.

The computer-implemented method of the first aspect, wherein identifying from the sets of 2D image slices the set of viable samples further comprising, for each sample: identifying foreground, background and multiple uncertain feature areas of the cellular structure in each of the 2D image slices; iteratively combining the foreground, background and uncertain feature areas of the 2D image slices to generate a single 2D image of the cellular structure; and selecting the sample for the viable sample set based on the quality of the single 2D image.

As an option, the multiple uncertain feature areas comprise multiple uncertain foreground features and multiple uncertain back features.

The computer-implemented method of the first aspect, wherein the cellular structure comprises one or more from the group of: cellular spheroid structures; vesicule; organoid; and any other suitable cellular structure.

The computer-implemented method of the first aspect, wherein the plate comprises a plurality of wells with a sample of the cellular structure within each well.

According to a second aspect, there is provided an apparatus comprising a processor, a memory unit and a communication interface, wherein the processor is connected to the memory unit and the communication interface, wherein the processor and memory are configured to implement the computer-implemented method according to the first aspect, combinations thereof, modifications thereto, and/or as herein described.

According to a third aspect, there is provided a computer-readable medium comprising data or instruction code, which when executed on a processor, causes the processor to implement the computer-implemented method according to the first aspect, combinations thereof, modifications thereto, and/or as herein described.

According to a fourth aspect, there is provided a tangible computer-readable medium comprising data or instruction code for predicting toxicity of one or more compounds applied to a plurality of samples of a cellular structure in an in-vitro microscopy assay, which when executed on one or more processors, causes at least one of the one or more processor(s) to perform at least one of the steps of the method of: receiving a set of images associated with the plurality of samples; inputting each image of the set of images to a first ML model configured for predicting phenotype features of the cellular structure within the sample associated with said each image; inputting each of the predicted phenotype features associated with each sample to a second ML model configured for predicting a lower dimensional phenotype feature embedding of said each sample; comparing the distance between lower dimensional phenotype feature embedding of said each sample that of a sample with compound having a known toxicity; and outputting, for each sample, an indication of the toxicity of said each sample and applied compound thereto based on said comparison.

According to a fifth aspect, there is provided a system comprising: a receiver module configured for receiving a set of images associated with the plurality of samples; a first ML model module configured for inputting each image of the set of images to a first ML model configured for predicting phenotype features of the cellular structure within the sample associated with said each image; a second ML model module configured for inputting each of the predicted phenotype features associated with each sample to a second ML model configured for predicting a lower dimensional phenotype feature embedding of said each sample; a distance comparison module configured for comparing the distance between lower dimensional phenotype feature embedding of said each sample that of a sample with compound having a known toxicity; and output module configured for outputting, for each sample, an indication of the toxicity of said each sample and applied compound thereto based on said comparison.

In various implementations, a computer program instructions, optionally stored on a non-transitory computer readable medium which, when executed by one or more processors of a data processing apparatus, causes the data processing apparatus to carry out the the program instructions to cause the one or more processors to perform operations comprising one or more aspects of the above-and/or below-described implementations (including one or more aspects of the appended claims).

In various implementations, apparatus are disclosed that comprise a computer readable storage medium having program instructions embodied therewith, and one or more processors configured to execute the program instructions to cause the apparatus to perform operations comprising one or more aspects of the above-and/or below-described implementations (including one or more aspects of the appended claims). The apparatus may comprise one or more processors or special-purpose computing hardware.

### BRIEF DESCRIPTION OF THE DRAWINGS

So that the specification may be more easily understood, embodiments thereof will now be described by way of example only, with reference to the accompanying drawings in which:

FIG. 1a illustrates an example toxicity prediction system for predicting toxicity of compounds applied to cellular structures of samples of a microscopy assay according to some embodiments of the specification;

FIG. 1b illustrates an example toxicity prediction process for use in the system of FIG. 1a according to some embodiments of the specification;

FIG. 2 illustrates an example neural network for predicting a phenotype embedding of a cellular structure in a sample of a microscopy assay of FIG. 1a according to some embodiments of the specification;

FIG. 3a illustrates an example assay plate with negative and positive control groups of samples for training a deep learning toxicity model and a test group of samples for input to the trained DL toxicity model according to some embodiments of the specification;

FIG. 3b illustrates an example unsupervised training process for a deep learning toxicity model using the negative and positive control groups of samples of FIG. 3a according to some embodiments of the specification;

FIG. 3c illustrates an example of predicting toxicity of compounds using the trained deep learning toxicity model of FIG. 3b according to some embodiments of the specification;

FIG. 4a illustrates another example assay plate with negative and positive control groups of samples for training a deep learning toxicity model and a test group of samples for input to a trained DL toxicity model according to some embodiments of the specification;

FIG. 4b illustrates an example distance matrix of negative and positive control samples of the trained DL model of FIG. 4a according to some embodiments of the specification;

FIG. 4c illustrates another example distance matrix of negative and positive control samples and test samples for predicting toxicity of compounds of the test samples using the trained DL toxicity model of FIG. 4b according to some embodiments of the specification;

FIG. 4d illustrates an example of a conventional toxicity prediction methodology used on a set of 8 compounds applied to samples of a cellular structure;

FIG. 4e illustrates an example of conventional toxicity prediction results for a set of compounds from the conventional toxicity prediction methodology of FIG. 4d;

FIG. 4f illustrates an example of the toxicity prediction results of the trained DL toxicity model for the same set of compounds as FIG. 4e;

FIG. 5a illustrates an example of a quality control image analysis process for selecting and pre-processing the microscopy images of cellular structures prior for use in training a DL toxicity model and/or as input to a trained DL toxicity model for predicting toxicity of compounds applied to said cellular structures according to some embodiments of the specification;

FIG. 5b illustrates an example of a first quality control process of a first quality control apparatus for use with the quality control image analysis process of FIG. 5a selecting a first set of viable images of samples for analysis according to some embodiments of the specification;

FIG. 5c illustrates an example of an image analyser of the first quality control apparatus for use with the first quality control process of FIG. 5b according to some embodiments of the specification;

FIG. 5d illustrates an example of the first quality control apparatus for use with the first quality control process of FIG. 5b according to some embodiments of the specification;

FIG. 5e illustrates an example of a second quality control process of a second quality control apparatus for use with the quality control image analysis process of FIG. 5a in pre-processing and selecting a final set of viable images of samples for analysis according to some embodiments of the specification;

FIG. 5f illustrates another example of the second quality control process of the second quality control apparatus control for use with the quality control image analysis process of FIG. 5a in pre-processing and selecting a final set of viable images of samples for analysis according to some embodiments of the specification;

FIG. 6a is a schematic illustration of a system/apparatus for performing methods described herein; and

FIG. 6b is a schematic illustration of another example system for performing methods described herein.

### Common reference numerals are used throughout the figures to indicate similar features.

### DETAILED DESCRIPTION

Various example implementations described herein relate to method(s), apparatus and system(s) for automatically, efficiently and reliably testing and predicting the toxicity of compounds applied to samples of a cellular structure in HTS microscopy assays. The toxicity prediction system receives a set of images of samples of a cellular structure, where at least a group of samples of the assay have been perturbed by one or more compounds under test. The received set of images are applied to a deep learning (DL) model trained and configured for predicting the toxicity of the compound's effects on the samples of cellular structure that have been perturbed. The DL model generates a high-dimensional phenotype representation of the cellular structure represented in each received image sample that is input. These are further processed into a lower-dimensional phenotype embedding focussing on features of the cellular structure associated with toxicity. A toxicity prediction for each image sample is output based on a distance or similarity metric for estimating the distance between each lower-dimensional phenotype embedding and a negative control lower-dimensional phenotype embedding, the negative control associated with a sample of the cellular structure not perturbed by any compounds under test.

The cellular structure of a sample is associated with an organ of a subject or patient and may be designed to mimic or simulate the organ. The cellular structure may be, without limitation, for example based on at least one from the group of: a cellular spheroid; a vesicule; an organoid; a cellular structure of an immortalised cell-line; and any other suitable cellular structure that mimics or simulates one or more processes of an organ of a subject or patient.

The toxicity prediction system is described herein with reference to, by way of example only but is not limited to, drug-induced hepatotoxicity or drug-induced liver injury (DILI), which is an acute or chronic response of the liver to a natural or manufactured compound. Up to 20-40% of DILI patients present a cholestatic and/or mixed hepatocellular/cholestatic injury pattern. The samples of the assay use a cellular structure associated with the liver, which follows the cholestasis into hepatocytes in-vitro. The type of cellular structure used is, without limitation, for example HepaRG® cells, which are terminally differentiated hepatic cells derived from a human hepatic progenitor cell line that retain many characteristics of primary human hepatocytes.

HepaRG cells are an immortalized cell line with 4 main features: 1—Full array of functions, responses, and regulatory pathways of primary human hepatocytes including: Phase I and II, and transporter activities consistent with those found within a population of primary human hepatocytes; 2—Form bile canaliculi; 3—Have the potential to express major properties of stem cells; 4—High plasticity & complete trans differentiation capacity. The cells can be used after a maturation of 7 days directly in an experiment assay plate. HepaRG cells may form cellular spheroids that mimic or simulate one or more cellular processes of the liver, which may be stained and/or fluoresced and imaged during in-vitro microscopy assays for downstream analysis.

Although the toxicity prediction system is described herein with reference to liver cellular structures/spheroids (e.g. HepaRG cell-line) and DILI, this is by way of example only and the specification is not so limited, it is to be appreciated by the person skilled in the art that the toxicity prediction system may be trained and applied to any type of cellular structure associated with any organ and/or any associated disease, any cell-line, array of cells, and/or wells of cellular samples with compounds that have been added to it. For example, the toxicity prediction system may be applied to samples of cellular structures that mimic an organ such as, without limitation, for example a lung, skin, kidneys, pancreas, liver, cardiac cellular structure/heart, neural cellular structures, and/or any other organ of the subject or patient. Such cellular structures may be used in samples with compounds applied thereto in in-vitro microscopy assays and the like and automatically analysed for toxicity by the toxicity prediction system.

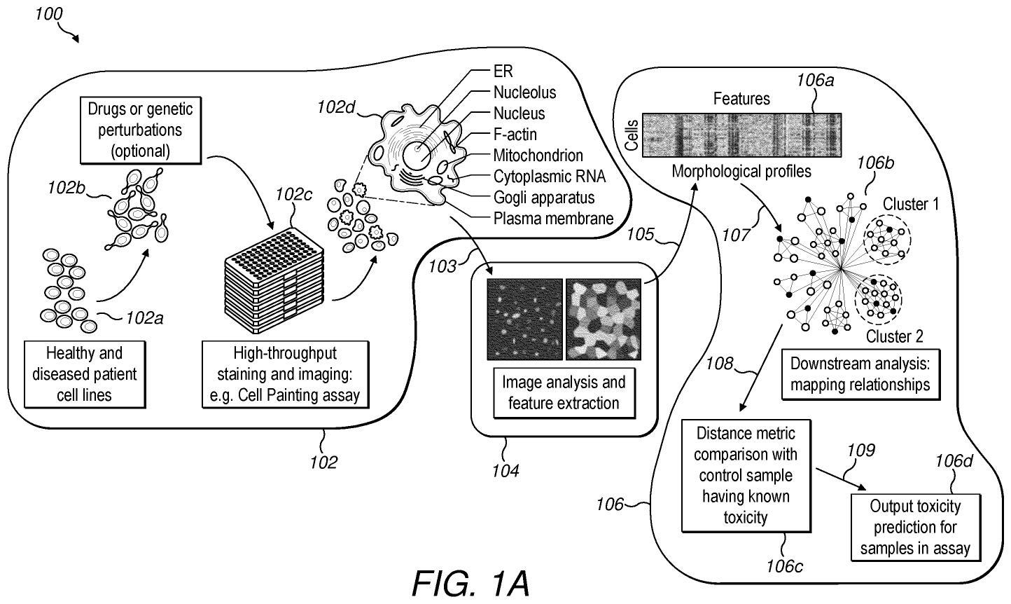

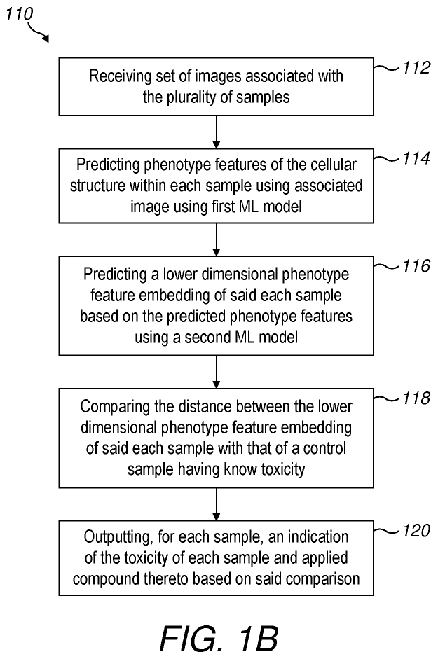

FIG. 1a illustrates an example toxicity prediction pipeline 100 for automatically predicting toxicity of one or more compounds applied to a plurality of samples of a cellular structure in an in-vitro microscopy assay. The toxicity prediction pipeline 100 includes an in-vitro HTS microscopy assay system 102, an imaging analysis system 104 and toxicity prediction system 106. The in-vitro HTS assay system 102 is configured to take a set of samples of a cellular structure 102a and applies one or more compounds or reagents 102b to the set of samples of the cellular structure 102a for input into a set of well samples in microscopy assay plates 102c for HTS staining and microscopy assay imaging 102d. In HTS staining and microscopy assay imaging 102d, the sample wells may be treated/stained with a fluorescence reagent/compound for emphasising the cellular structure of each sample. For example, the treated/stained cellular structure such as, for example, the spheroid structure, vesicules, and/or nuclei may be imaged by a microscopy imaging system. Thus, the in-vitro HTS microscopy assay system 102 is configured to output a set of well image samples 103. Each well sample image in the set corresponding to each sample of the cellular structure and compound applied thereto in the set of well samples of the assay.

The imaging analysis system 104 may be configured to receive the set of well sample images and perform image pre-processing and/or analysis to identify which samples from the set of well samples are viable for further downstream analysis such as, for example, toxicity prediction of the compounds applied to the samples of the set of well samples. One or more image processing and/or machine learning algorithms may be applied to identify the viability of each well sample image based on any detected image artifacts and/or imaging defects and the like, and/or for enhancing or emphasising the cellular structures of interest within each viable well sample image of the set of well samples. As a result, a set of viable well sample images 105 may be output by the imaging analysis system 104 for further downstream analysis. In essence, the set of viable well sample images 105 may be any suitable set of images of a cellular structure from the set of well samples of the assay that sufficiently describes the cellular structure of the sample for automated analysis.

The toxicity prediction system 106 is configured to receive a set of images of cellular structures 105 derived from a plurality of samples of cellular structures with one or more compounds applied thereto during an in-vitro microscopy assay. In this case, the received set of sample images 105 may be a set of viable well sample images 105 output from the imaging analysis system 104. However, any suitable set of images that sufficiently describe the cellular structure of a set of well samples with compounds applied thereto may be received by the toxicity prediction system 106. For example, the set of well image samples 103 output from the in-vitro HTS microscopy assay system 102 may be used, as long as, the images are sufficiently free of artifacts and/or defects and components of the cellular structure of each sample is analysable.

In the toxicity prediction system 106, the received set of sample images 105 may each be input to a deep learning (DL) toxicity prediction model 106a-106c that is configured to predict the toxicity of each corresponding compound applied thereto in the assay from the in-vitro HTS microscopy assay system 102. The DL toxicity prediction model 106a-106c may be based on any one or more DL modelling technique/algorithms and/or machine learning (ML) technique/algorithms, which have been used in training the DL toxicity model to identify or predict whether each of the received sample images 105 indicates toxicity or not, even when a compound has not been applied to the cellular structure of one or more of the well samples. The one or more DL/ML techniques/algorithms may be based on supervised ML, unsupervised ML and/or semi-supervised ML algorithms and the like. However, for the task of training a DL toxicity prediction model 106a-106c, it has been found that supervised learning is difficult due to the limited number of labelled training datasets in relation to cellular structures that indicate toxicity or not depending on whether compounds have been applied or not. As a result, a combined supervised/unsupervised DL model training architecture may be used for training one or more component models of the DL toxicity prediction model 106a-106c.

For example, in the example of FIG. 1a, the DL toxicity prediction model 106a-106c may include a trained machine learning (ML) phenotype feature extraction (FE) model 106a for extracting phenotype features of the cellular structure from each received sample image of the received set of sample images 105. Supervised learning may be used for training the ML phenotype FE model 106a. The ML phenotype FE model 106a may be based on, without limitation, for example a neural network (NN) classifier that is trained using supervised training on readily available labelled/annotated training datasets for classifying images of cells, organoids, spheroids, cellular structures, and the like (e.g. classifying images of cellular structures to determine whether the cells are cancer or tumour cells or not). The NN classifier may be based on any NN structure such as, without limitation, for example a feed forward NN (FNN), recursive NN (RNN), artificial NN (ANN), convolutional NN (CNN), any other type of NN, modifications thereto, combinations thereof. Prior to the output classification layer or SoftMax output of the NN classifier, the phenotype representation of a cellular structure may be embedded by the high dimensional output of one of the hidden layers or full layers of the NN classifier. Rather than output the classification, the NN classifier is configured to output the embedding of the phenotype representation of the cellular structure from said hidden or full layer.

Typically, the phenotype representation embedding of an input image sample from the received image sample set 105 is a high dimensional representation of the phenotype features (e.g. for CNN type NN classifiers/models, the dimensionality may be in the order of 2024 or larger). The trained NN classifier may be used to output a high dimensional phenotype feature representation 107 of the cellular structure for each input well sample with compound applied thereto in the set of sample images 105. As an example, the neural network classifier may be based on a convolutional neural network (CNN) for classifying images of cellular structures (e.g. classifying images of cellular structures to determine whether the cells are cancer cells or not) in which, once trained, one of the final full layers of the CNN may be used as the phenotype feature representation for each of the received set of image samples 105 that are input thereto.

The ML phenotype embedding model 106a is coupled to a trained ML lower dimensional (LD) embedding model 106b, which is trained and configured for optimally embedding the high dimensional phenotype representation 107 of the predicted phenotype features of each received sample image of the received set of image samples 105 into a lower dimensional phenotype embedding 108 of a lower dimensional space for further analysis, where the lower dimensional phenotype feature embedding 108 includes those phenotype features associated with toxicity. Rather than using supervised training on the ML LD embedding model 106b, one or more unsupervised DL/ML techniques/algorithms is used to ensure the trained ML LED embedding model 106b outputs a LD phenotype embedding 108 representing phenotype features associated with toxicity of a cellular structure. The unsupervised DL/ML techniques/algorithms may be based on at least clustering algorithms or dimensionality reduction algorithms such as, without limitation, for example Support vector machines (SVM), Uniform Manifold Approximation and Projection (UMAP) or t-distributed Stochastic Neighbour Embedding (t-SNE) type algorithms, combinations thereof, modifications thereto and the like.

For example, the ML LD embedding model 106b may be trained using negative control and positive control samples included on the assay plate 102c in the in-vitro microscopy assay. That is, the assay plate 102c may include a negative control group of samples, which may be a first group of wells of the assay plate 102c in which the samples were not perturbed or had no compounds applied thereto, and a positive control group of samples, which may be a second group of wells of the assay plate 102c in which the samples were perturbed with a known compound with known toxicity. The assay plate 102c may also include a third group of wells of the assay plate 102c that includes samples of cellular structures with compounds applied thereto. Thus, the negative and control groups of images associated with the negative and positive control samples may be used to train the ML LD embedding model 106b in an unsupervised manner.

For example, the negative and control groups of images are input to the ML phenotype embedding model 106a, which outputs corresponding negative and positive high dimensional phenotype representations. Then an iterative optimisation of the parameters of the UMAP or t-SNE algorithms may be performed on the high dimensional negative and positive control phenotype representations that are output from the ML phenotype embedding model 106a, in which the differences between the resulting LD negative and positive control phenotype representation embeddings are maximised. The resulting optimised parameters may be used with the UMAP or t-SNE algorithms for dimensional reduction of the high dimensional phenotype representations 107 corresponding to the third group of well samples. The LD phenotype representations 107 may be output for comparison with the negative control (NC) LD phenotype representation using a suitable distance or similarity metric used by trained ML distance model 106c.

The ML LD embedding model 106b is coupled to a trained ML distance/prediction model 106c/106d configured for performing a distance or similarity metric comparison/estimate 109 between the LD phenotype representation of one of the image samples of a well and the negative control LD phenotype representation using a suitable distance or similarity metric in relation to the LD space of the LD phenotype representation. The ML distance model 106c may output data representative of each distance comparison estimate 109, which is input to ML prediction unit 106d for predicting the toxicity. It is noted that the LD space of the LD phenotype representation embedding output by the ML LD embedding model 106b may still be considered a high dimensional space in which the Euclidean distance metric or similarity metrics break down and/or cannot be reliably used. Thus, high dimensional distance or similarity metrics may be used based on, without limitation, for example Wasserstein distances and/or any other high dimensional distance metric or similarity metric. The ML distance/prediction model 106c/106d outputs a toxicity prediction as a probability based on the distance comparison 109 (e.g. Wasserstein distance comparison) between each LD phenotype representation of a sample and the negative control LD phenotype representation.

FIG. 1b illustrates an example toxicity prediction process 110 for use by the toxicity prediction system 106 in predicting toxicity of one or more compounds applied to a plurality of samples of a cellular structure in the in-vitro microscopy assay pipeline 100 of FIG. 1a. The toxicity prediction process 110 includes the steps of: In step 112, receiving a set of images associated with a plurality of samples based on the output of an in-vitro microscopy assay. Each sample image includes image data that sufficiently describe the cellular structure of the associated sample for automated processing and analysis. In step 114, inputting each image of the set of images to a first ML model configured for predicting phenotype features of the cellular structure within the sample associated with said each image. In step 116, inputting each of the predicted phenotype features associated with each sample to a second ML model configured for predicting a lower dimensional phenotype feature embedding of said each sample. In step 118, comparing the distance between the lower dimensional phenotype feature embedding of said each sample with that of a sample applied with a compound having a known toxicity (e.g. negative control sample). In step 120, outputting, for each sample, an indication (e.g. probability) of the toxicity of said each sample and applied compound thereto based on said comparison.

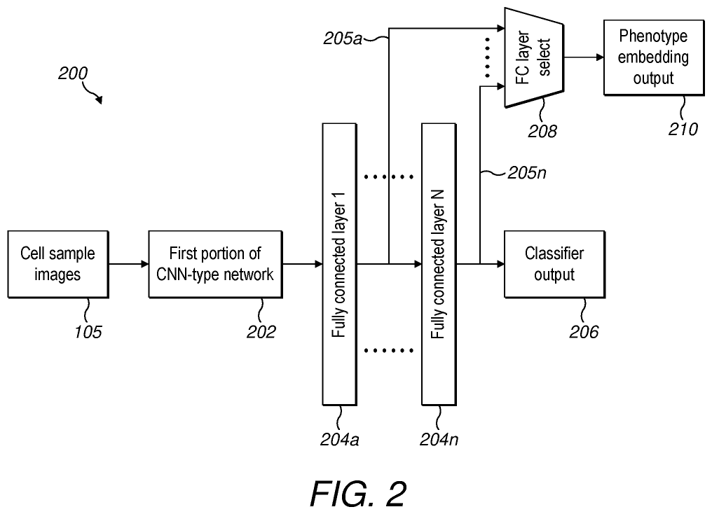

FIG. 2 illustrates an example neural network classifier 200 for use in ML phenotype FE model 106a of toxicity prediction system 106 of FIG. 1a. The NN classifier 200 is configured for predicting or outputting a phenotype representation/embedding from an image of a cellular structure in a sample of a microscopy assay of FIG. 1a. The NN classifier 200 is based on a CNN-type architecture that includes a first portion of a CNN-type network 202 followed by one or more fully connected layers 2104a-204n and a classifier output layer 206. One or more of the outputs 205a-205n of the fully connected layers 204a-204n may tapped and/or selected 208 for output as a phenotype representation embedding 210 associated with the input sample image representing the cellular structure. As an example, the NN classifier 200 may be a 50-layer RESNET CNN architecture with multiple fully connected layers (e.g. RESNET50® or VGG® and the like).

The NN classifier 200 is trained on image data containing cellular structures in relation to a classification task, where relevant phenotypic information of a cellular structure may be extracted from the output of one of the hidden or fully connected layers of the trained NN classifier 200. The NN classifier 200 may be pre-trained for a classification task using labelled image data associated with cell types/structures. For example, the NN classifier 200 may be trained using a labelled training image dataset to classify images as being, without limitation, for example cancerous or not cancerous, identification of cells/cell structures, and/or other diseases affecting cellular function/structure.

In essence, when training for the classification task, the layers within the CNN architecture of the NN classifier 200 start to “recognize” cell primitives, cell agglomerations, how cells form tissue regions and the like, towards recognizing larger cellular macrostructures, which can be leveraged to form, in the full layers 204a-204n, a high dimensional phenotype representation embedding of the cellular structures present in an input image of a sample of a cellular structure. The classification task or output 206 is used only for training the NN classifier 200. As another example, the NN classifier 200 may be trained on labelled cell image data from ImageNet and then fine-tuned using a limited number of training image data items associated with cell data and/or stained/fluorescence cell imagery from microscopy assays and the like.

The NN classifier 200 architecture may be configured to have multiple fully connected layers 204a-204n prior to the output classification 206. One of the fully connected layers 204n is selected out of all of the fully connected layers 204a-204n based the one that is determined to focus on, in terms of deep representation, of cellular structures. As an option, the toxicity prediction system 106 may perform an automated optimization of each of the ML models 106a-106c to determine which of the fully connected layers 204a-204n of the trained NN classifier 200 provides the best high dimensional phenotype representation output that highlights toxic effects or toxicity in the cellular structures.

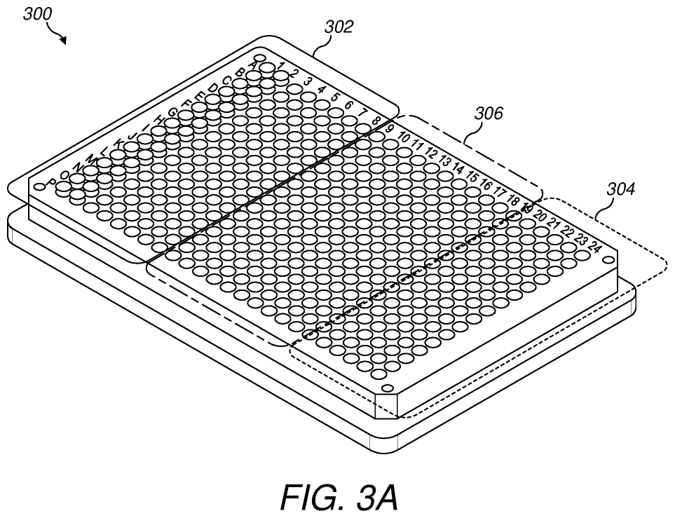

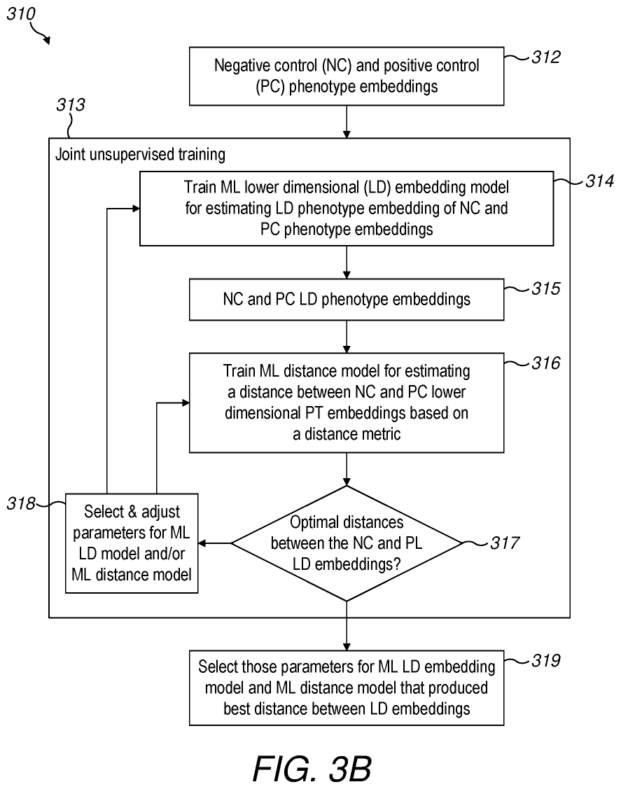

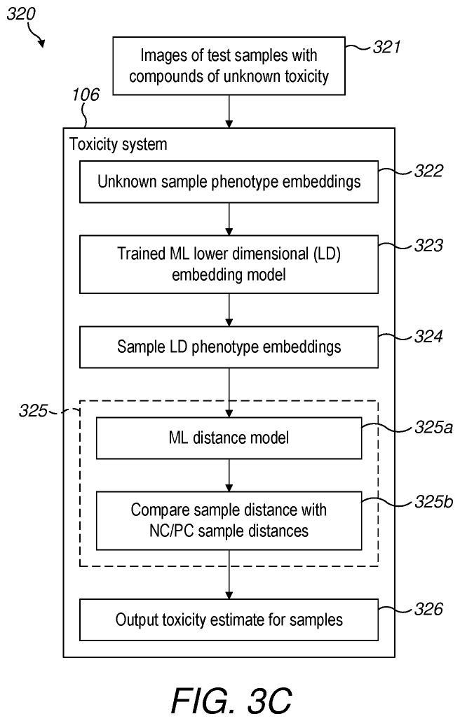

FIG. 3a an example assay plate 300 with negative and positive control groups of sample wells 302 and 304 in which the samples therein are imaged during an in-vitro microscopy assay for use in training a deep learning (DL) toxicity model 106. The assay plate 300 also includes a test group of sample wells 306 in which the samples therein are imaged during in-vitro microscopy assay for input to the trained DL toxicity model for predicting toxicity of any compounds applied to said test group of sample wells 306. The negative control group of sample wells 302 includes samples that have not been perturbed by a compound under test, or have been perturbed by a compound that is non-toxic. For example, in in-vitro microscopy assays using liver spheroids, Dimethyl sulfoxide (DMSO) may be applied to the NC samples (e.g. 0.6% DMSO). The positive control group of sample wells 304 includes samples that have been perturbed by a compound with a known toxicity in relation to the cellular structure used in the samples. For example, in in-vitro microscopy assays using liver spheroids, the PC samples may be perturbed using Chlorpromazine (CPZ) at a concentration that gives a toxic effect (e.g. 400 μM).

The DL toxicity model 106 is trained and configured to first extract phenotypical features of the cellular structure of each sample in the sample image and then estimate the phenotypical distance between the extracted phenotypical features and the phenotypical features of associated with samples in the negative control group of sample wells 302. The microscopy images of negative control samples and positive control samples in the negative and positive control group of sample wells 302 and 304, respectively, are used with unsupervised DL/ML techniques for training the DL toxicity model 106 to estimate toxicity of the compounds of each sample using the phenotypical distance between the extracted phenotypical features and the phenotypical features of associated with samples in the negative control group of sample wells 302. For example, the negative control samples are used to build an average and deep phenotypical representation embedding of the negative control samples (e.g. first 20 wells). Once, the average and deep phenotypical representation embedding of the negative control samples has been built, this can be used as a reference for determining the distance of the low dimensional embeddings of subsequent test samples.

In this example, the assay plate 300 has an array of wells in rows and columns wells (e.g. cols A-P and rows 01-24), which are mapped to the group of negative control sample wells 302, positive control sample wells 304 and test sample wells 306. In this example, the assay plate 300 has an array of 16 columns and 24 rows of wells, or a total of 384 wells. Although the assay plate 300 illustrated the group of negative control wells 302 as being mapped to columns A-P and rows 1-8, the group of positive control wells 304 as being mapped to columns A-P and rows 20-24, and the group of test sample wells 306 as being mapped to columns A-P and rows 9-19, this is by way of example only and the specification is not so limited, it is to be appreciated by the skilled person that any mapping can be defined for assigning a mapping between the sample wells of the assay plate 300 and the group of negative control sample wells 302, positive control sample wells 304 and test sample wells 306. The mapping of samples to wells in the assay plate 300 may be automatically defined by the software management system of the HTS in-vitro microscopy assay system 102 depending on the number of samples required in each group of negative control sample wells 302, positive control sample wells 304 and test sample wells 306. For example, the samples in the test group of sample wells may have one or more compounds at varying concentrations applied thereto. The HTS in-vitro microscopy assay system 102 may be programmed to map the type of compounds and the concentrations onto the assay plate 300, which depends on how the number of compounds, the number of different concentrations, and number of replicates that are to be tested and the like.

FIG. 3b illustrates an example unsupervised training process 310 for training a deep learning toxicity model including the ML low dimensional (LD) embedding model 106b and ML distance model 106c of the toxicity prediction system 106 of FIG. 1a. Training the DL toxicity model uses the negative and positive control groups sample wells 302 and 304 of samples of FIG. 3a. The unsupervised training process 310 includes the following steps of:

In step 312, the training process 310 receives negative control (NC) and positive control (PC) phenotype representation embeddings associated the NC and PC samples corresponding to the groups of NC and PC sample wells 302 and 306 of plate 300. Images based on the NC and PC samples corresponding to the groups of NC and PC sample wells 302 and 306 of plate 300 may be input to the ML phenotype feature extraction model 106a of FIG. 1a, which may be based on the NN classifier 200 of FIG. 2 and has already been pre-trained as described with reference to FIGS. 1a-2. Given this, the ML phenotype feature extraction model 106a outputs corresponding NC and PC phenotype representation embeddings 107. The NC and PC phenotype representation embeddings 107 are high dimensional embeddings (e.g. 2024 elements per image).

In step 313, a joint unsupervised training of the ML LD embedding model 106b and the ML distance/prediction model 106c/106d is performed based on the following steps of: In step 314, performing unsupervised training of the ML LD embedding model 106b for estimating LD phenotype embeddings 108 representing the toxicity features associated with the NC and PC phenotype representation embeddings. Unsupervised training may be performed by iteratively optimising the ML LD embedding model 106b over a set of parameter ranges associated with the ML technique (e.g. UMAP or t-SNE) used to configure the ML LD embedding model 106b. The criterion of the ML technique for adjusting or generating the ML LD embedding model 106b is to find the subset of parameters that maximises the differences between the NC embeddings and the PC embeddings using the input high dimensional NC and PC phenotype representation embeddings over the set of parameter ranges. In each iteration, a subset of parameters is selected from the set of parameters ranges and applied to the ML technique (e.g. UMAP or t-SNE), which adjust or generates the ML LD embedding model 106b for outputting LD NC and PC phenotype embeddings.

As an example, the input high dimensional NC and PC phenotype representation embeddings 107 output from the ML phenotype FE model 106a is a high dimensional vector per image (e.g. each embedding may be 2024 elements per image). The ML phenotype FE model 106a has not been trained specifically for toxicity prediction but rather for identifying biological structure or phenotypical representation of the cellular structure within each image of a sample. Thus, the resulting high dimensional NC and PC phenotype representation embeddings describe, as much as possible within the number of dimensions (e.g. 2024), the phenotypical representation of each corresponding cellular structure. The UMAP algorithm may be used to generate the ML LD embedding model 106b to not only reduce the dimensionality of the high dimensional NC and PC phenotype representations, but to also ensure phenotypical representations associated with toxicity are retained or focussed on within the resulting LD NC and PC embedding vectors 108 (e.g. each embedding may be 64 elements per image) that are output from the ML LD embedding model 106b. This is performed by the UMAP algorithm finding the optimal set of parameters, within the set of parameter ranges, that maximise the differences between high dimensional NC and PC phenotype representation embeddings. The ML LD embedding model 106b is trained to find relevant biological structure associated with toxicity within each image in an unbiased manner without knowing the type of cells or application thereto. The ML LE embedding model 106b may use the LD embedding (e.g. 64 dimensions in the embedding) to represent the entire group of negative controls by taking a simple mean profile of all negative controls. This may be used by the ML distance/prediction model 106c/106d for determining a prediction of toxicity based on using a distance or similarity metric to estimate the distance or similarity between a LD embedding of a test sample from the group of test sample wells 306 and the average LD NC embedding of the NC samples from the group of NC sample wells 302.

In step 315, the ML LD embedding model 106b outputs the NC and PC LD phenotype embeddings 108, where each embedding includes phenotypical information of the corresponding cellular structure in relation to toxicity. The NC and PC LD phenotype embeddings are used as input to step 316 for training the ML distance model 106c.

In step 316, performing unsupervised training of the ML distance model 106c for estimating, based on a suitable distance or similarity for high dimensional vectors metric (e.g. Wasserstein distance), for maximising the distance between the NC and PC LD phenotype embeddings. This means that the trained ML distance model 106c can determine the distance between NC LD phenotype embeddings and LD phenotype embeddings of test samples 109 for determining the probability or an indication of whether the compound associated with each test sample is toxic or not. The unsupervised training may be performed by iteratively optimising the ML distance model 106c over a set of parameter ranges associated with the ML distance algorithm/technique (e.g. Wasserstein distance algorithms such as Earth Mover's distance and/or Sinkhorn distance algorithms) used to configure the ML distance model 106c. The criterion of the ML distance algorithm/technique for adjusting or generating the ML distance model 106c is to find the subset of parameters of the ML distance algorithm that maximises the distance between the set of NC LD embeddings and the set of PC embeddings, but minimises the distance between embedding within the set of NC LD embeddings and minimises the distance between embeddings within the set of PC LD embeddings. In each iteration, a subset of parameters is selected from the set of parameters ranges for the ML distance algorithm and applied to the ML distance algorithm (e.g. Earth Mover's distance algorithm or Sinkhorn algorithm), which adjusts or generates the ML distance model 106c/106d for outputting an indication of toxicity between an LD phenotype embedding and the set of NC LD embeddings.

In step 317, it is determined whether the optimal distances between the sets of NC and PC LD embeddings have been attained for the set of parameter ranges of the ML distance model 106c. This may also include determining whether the differences between the NC LD embeddings and PC LD embeddings have been maximized based on the sets of parameters tested so far. If this is the case or if there are no more combinations of parameters from the set of parameters for either the ML LD embedding model 106b or ML distance model 106c that may be used, then the process 310 proceeds to step 319. Otherwise, the process proceeds to step 318 for further parameter selection and adjustment/training of either the ML LD embedding model 106b and/or the ML distance model 106c.

In step 318, where further parameters are selected from the set of parameters associated with the ML LD embedding model 106b for further training thereof, where the process 310 proceeds to step 314. Similarly, further parameters may be selected from the set of parameters associated with the ML distance model 106c for further training thereof, where the process 310 proceeds to step 316.

In step 319, the subset of parameters over the set of parameters associated with the ML LD embedding model 106b that maximizes the differences between the NC and PC high dimensional phenotype embeddings are selected for use by the ML LD embedding model 106b in outputting LD phenotype embeddings in relation to high dimensional phenotype embeddings corresponding to the test samples of the group of test sample wells 306. As well, the subset of parameters over the set of parameters associated with the ML distance model 106c that maximized the distance (e.g. Wasserstein distance) between the sets of NC and PC LD phenotype embeddings, but which also minimized the distance within each set of NC and PC LD phenotype embeddings are selected for use by the ML distance/prediction model 106c/106d in outputting an estimate of the toxicity based on the distance of the LD phenotype embeddings corresponding to the test samples of the group of test sample wells 306 and the set of NC phenotype embeddings or average NC phenotype embedding.

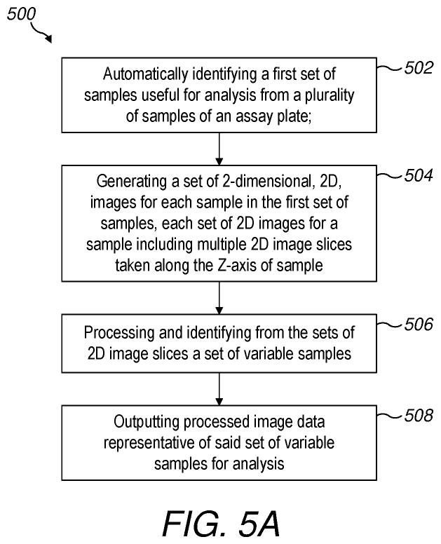

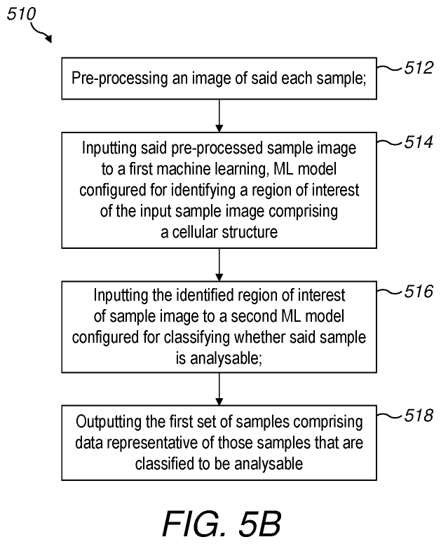

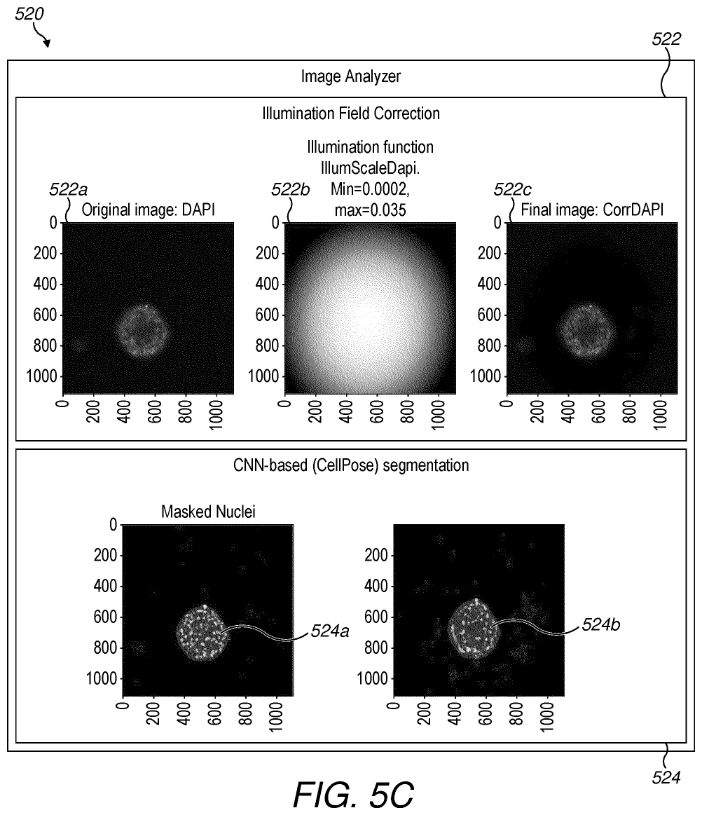

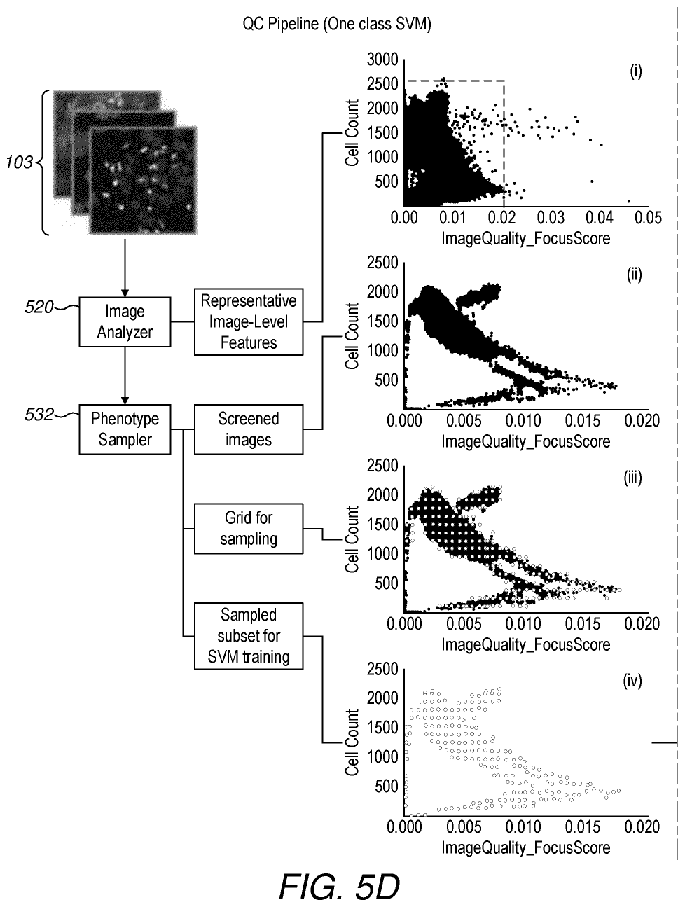

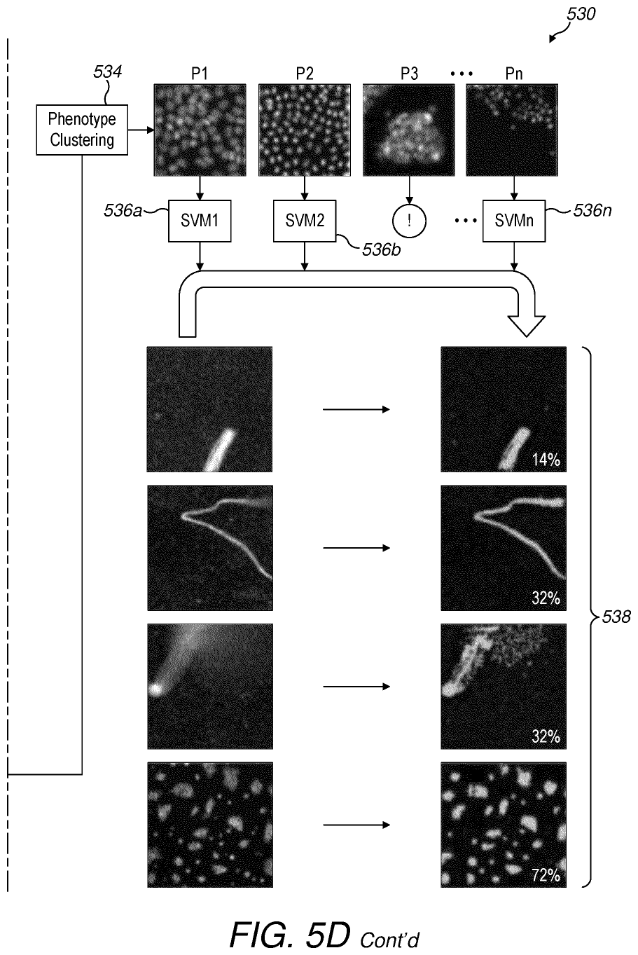

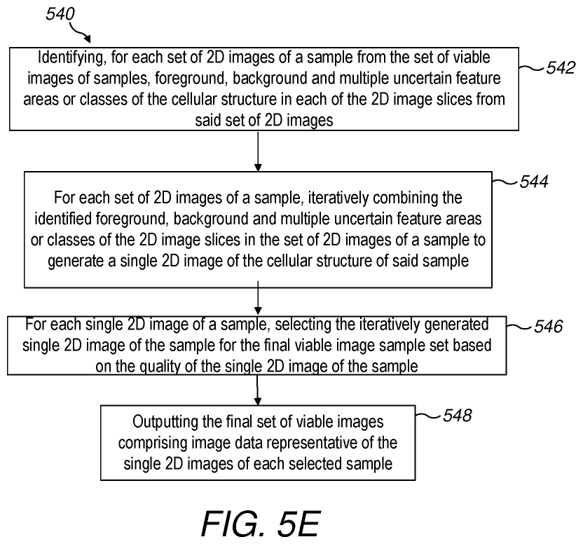

FIG. 3c illustrates the toxicity prediction process 320 for predicting the toxicity of compounds using the trained deep learning toxicity model of FIG. 3b. From step 319 of toxicity training process 310 of FIG. 3b, the ML LD embedding model 106b and ML distance model 106c and prediction model 106d are configured based on the output selected parameter sets 310. The DL toxicity model of FIG. 3b includes the ML LD embedding model 106b trained as described with reference to FIG. 3b and the ML distance model 106c trained as described with reference to FIG. 3b. The toxicity prediction system 106 includes the ML phenotype model 106a as described with reference to FIGS. 1a to 2 and the DL toxicity model of FIG. 3b, which is used to predict toxicity of one or more compounds applied to a plurality of test samples of a cellular structure in an in-vitro microscopy assay. The plurality of test samples in the group of sample test wells 306 of FIG. 3a may be captured by a microscopy imager during the in-vitro microscopy assay. During the in-vitro microscopy assay a set of one or more compounds may be applied to the test samples, i.e. one compound per test sample. These may be processed and/or enhanced using imaging system 104 of FIG. 1a or the quality control system and processes of FIGS. 5a to 5f. The resulting images of the test samples may then be input to the toxicity prediction system 106 for predicting the toxicity of the compounds that were applied to the test samples. The toxicity prediction process 320 includes the steps of:

In step 321 receiving a set of images associated with one or more test samples with compounds applied thereto from the group of sample test wells 306 of an in-vitro microscopy assay. The compounds may have a known or unknown toxicity on the cellular structure within the corresponding test samples. Each image of a test sample includes image data that sufficiently describes the cellular structure of the associated test sample with compound applied therein for automated processing and analysis. In step 322, inputting each image of the set of images to the trained ML phenotype feature extraction model 106a or 200 of FIG. 1a or 2. The trained ML phenotype feature extraction model 106a or 200 outputs, for each input image of a test sample, a high dimensional phenotype representation of the cellular structure of the test sample that is within the input image of the test sample. The output high dimensional phenotype representation of each test sample is then applied to the trained ML LD embedding model 106b. In step 323, inputting each of the high dimensional phenotype representations of each test sample to the trained ML LD embedding model 106b for outputting low dimensional phenotype embeddings of each test sample. In step 324, the LD phenotype embeddings of each test sample are passed to the trained ML distance model 106c.

In step 325, the ML distance model 106c receives each LD phenotype embedding 108 of each test sample and outputs a distance estimate 109 between the LD phenotype embedding of each test sample and the set of NC LD phenotype embeddings. In step 325a, the ML distance model 106c may be configured to output a distance or similarity estimate 109 between the LD phenotype embedding of each test sample and the average NC LD embedding of the set of NC LD phenotype embeddings. This may include, in step 325b, comparing the distance between the LD phenotype embedding of a test sample with that of the average NC LD embedding of the set of NC LD embeddings. From step 325, the ML distance/prediction model 106c/106d may output an indication or probability associated with the distance each of the LD phenotype embeddings is to the NC LD embedding (i.e. determined how far away the LD phenotype embedding is from the non-toxic NC LD phenotype embedding). In step 326, outputting, for each test sample, an indication (e.g. probability) of the toxicity of said each test sample and applied compound thereto based on said comparison of the ML distance model 106c.

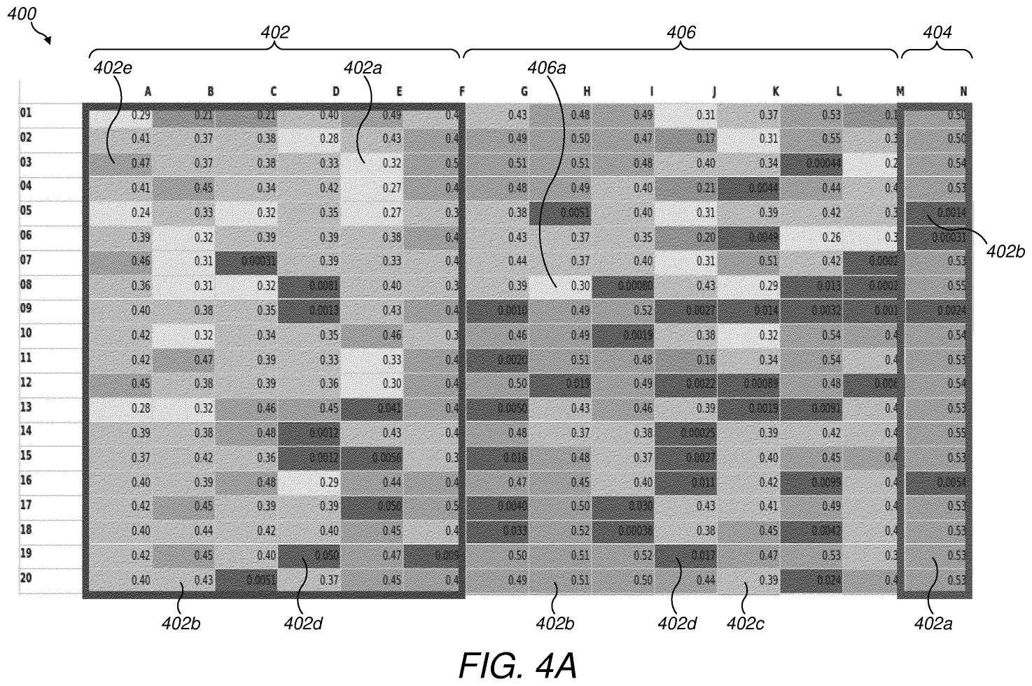

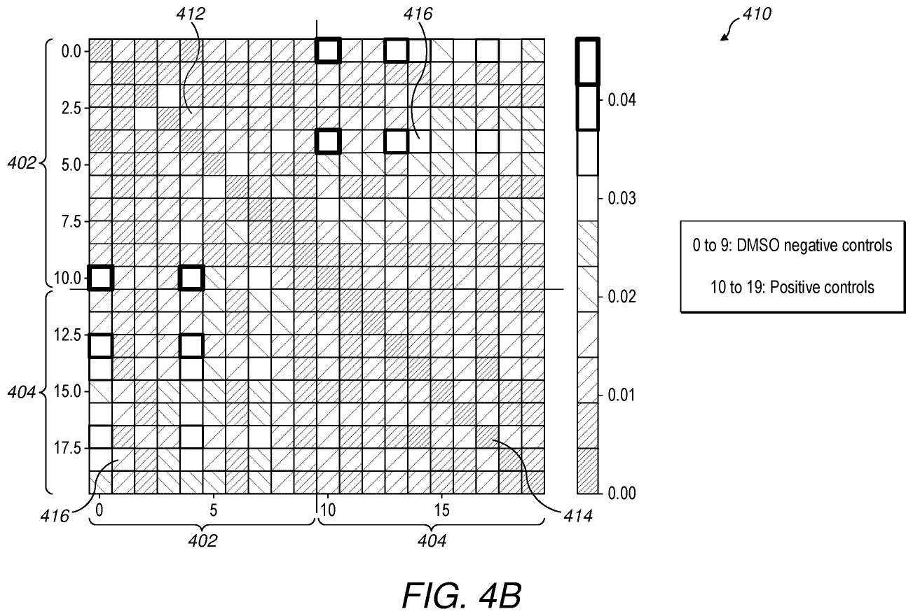

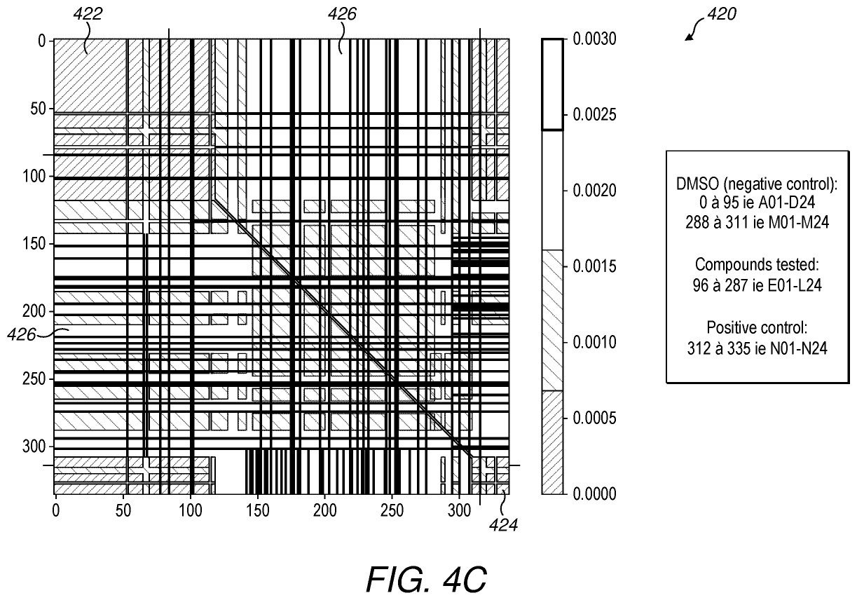

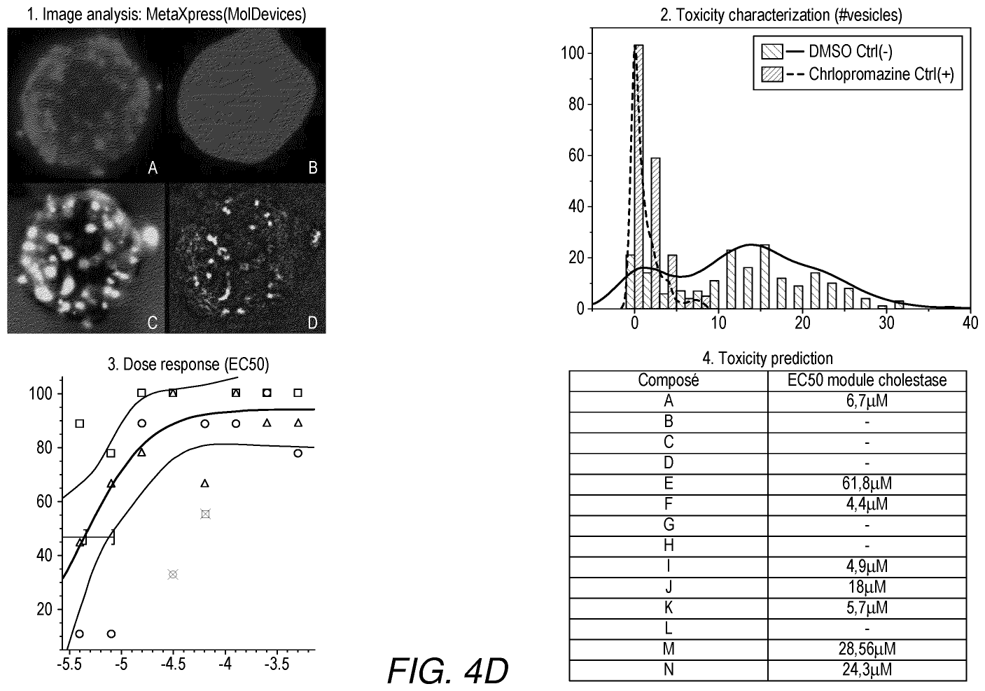

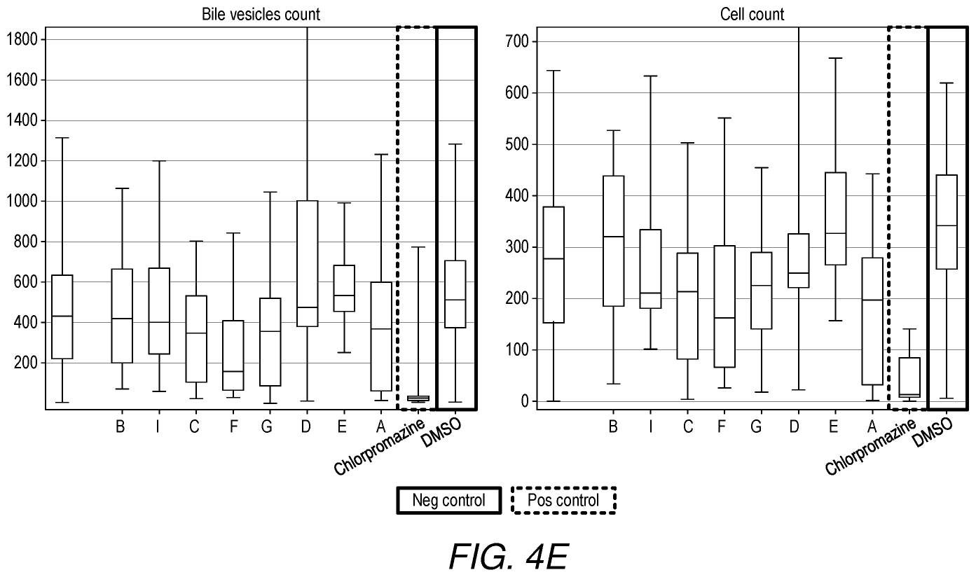

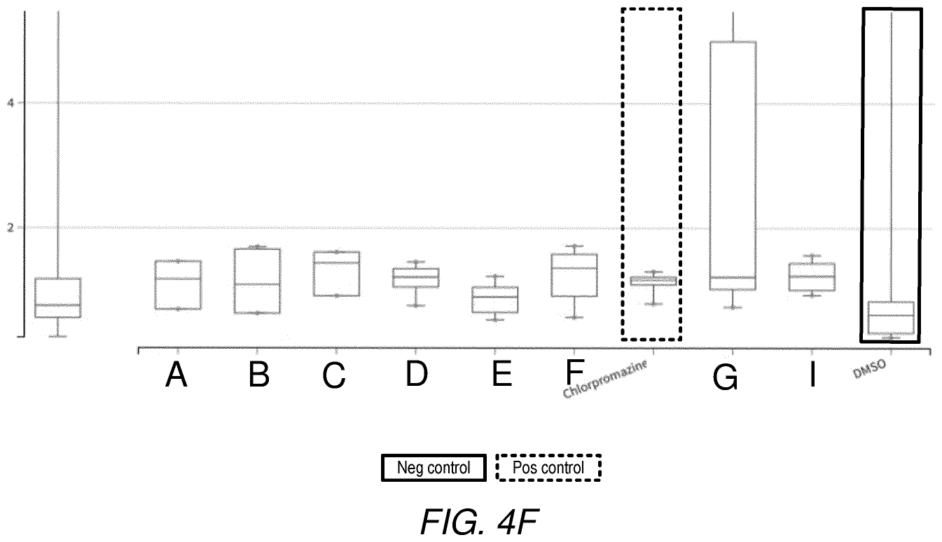

FIG. 4a is a schematic diagram illustrating another example assay plate 400 with negative and positive control groups of samples 402 and 404 for use in training the deep learning (DL) toxicity model of the toxicity prediction system 106a and models as described with reference to FIGS. 1a, 2 and 3a-3b and a test group of samples 406 for input to the trained DL toxicity model of the toxicity prediction system 106.

In this example, the HepaRG liver cell-line is used for determining the toxicity of compounds in the liver. The each of the sample wells of the sample plate 400 is populated with the HepaRG cellular structure, where the in-vitro micro assay system 102 of FIG. 1a is configured to evaluate the cell cholestasis effects of compounds using specific fluorescence substrates (e.g. Carboxy-DCFDA (5-(and-6)-Carboxy-2′,7′-Dichlorofluorescein Diacetate) or CDFDA). CDFDA is a reagent that passively diffuses into cells. It is a florescence substrate for use by the imager to capture the accumulation of CDF in canaliculi biliary in the image of each sample. This enables evaluation of whether a compound provokes cholestasis, which has been observed to occurs when canaliculi biliary of the cellular structure in an image disappears. However, the toxicity prediction system 106 performs further unbiased processing to take into account other unobserved changes to the cellular structure when determining toxicity of compounds.

The samples with the HepaRG cellular structure in the negative control group of samples 402 only have a buffer (e.g. DMSO) applied to them, which has no toxicity effect. The samples with the HepaRG cellular structure in the positive control group of samples 404 have a reference compound (e.g. CPZ @ 60 micro moles) with known toxic effect that is known to be toxic to liver cells and triggers cholestasis. The samples with the HepaRG cellular structure in the test group of samples 406 have various different compounds applied at different concentrations and replicates. In this example, each compound may be represented in the assay plate 400 by 8 doses in which each dose is represented in the assay plate 400 by 3 replicates. This is to ensure there are enough replicates that after applying quality control to the images of the test samples then there will be at least one replicate for every compound and every dose that has a viable image of the test sample for further downstream analysis.

Images of each of the sample wells of the assay plate 400 are captured and assessed for viability in relation to further downstream analysis by the toxicity prediction system 106. This may be performed by image system 104 of FIG. 1a and the image quality control systems and/or processes of FIGS. 5a to 5e. For example, a quality control model may be trained to automatically assess the viability of each well sample by classifying the associated image of the test sample in the well. Each well sample is classified with a probability indicating whether the well sample is good quality of bad quality. The higher the probability value the better the quality, the lower the probability value the worse the quality. A threshold probability may be used for determining the higher quality sample wells that are viable. In this example, a probability value of about 0.1 was determined to result in viable sample images that could be passed through to the toxicity prediction system 106 for toxicity training and/or toxicity prediction. The light grey shaded areas, for example images of the samples in wells 402a-402c, 404a, 406a-406c, represent the samples in the NC, PC and test sample wells are indicative of viable samples for further downstream analysis. The darkest grey shaded areas, for example images of the samples in wells 402d, 406b and 404d, represent the samples in the NC, PC and test sample wells that are indicative of non-viable samples that have too many artifacts for analysis and may be discarded. Thus, a set of viable sample images from each of the groups of NC, PC and test samples 402, 404 and 406 may be used for further downstream analysis. The set of viable images from the group of NC samples 402 and the group of PC samples 404 are used to train, in an unsupervised way the DL toxicity model, which includes training the ML LD embedding model 106b and ML distance model 106c/106d of FIGS. 1a, as described with reference to FIGS. 1a to 3b. The toxicity prediction system includes the ML phenotype feature extraction model 106a and the DL toxicity model, which includes the ML LD embedding model 106b and ML distance model 106c/106d of FIGS. 1a.

FIG. 4b illustrates an example distance matrix of negative and positive control samples of the trained DL toxicity model trained based on the unsupervised training process of FIGS. 1a and 3a using the viable NC and PC samples from NC and PC sample groups 402 and 406 on the assay plate 400 of FIG. 4a. Once the viable images of the NC and PC samples have been passed through the ML phenotype feature extraction model 106a or 200 of FIGS. 1a-2, the output sets of NC and PC high dimensional phenotype embeddings are used as input to the UMAP algorithm to train and generate the ML LD embedding model 106b as described with reference to FIG. 3b.

For example, the set of parameters that define the hyper parameters of the UMAP algorithm are iteratively optimized using a grid search, where different values of hyperparameters from the set of parameters are iteratively applied to determine the best combination of UMAP parameters that maximize as much as possible the difference between the set of NC low dimensional phenotype embeddings and the set of PC low dimensional embeddings. Given the LD phenotype embeddings are still of a high dimension (e.g. 64 elements) the Euclidean, Manhattan and other standard distance metric cannot be applied, so instead the Wasserstein distance is used.

As described with reference to FIGS. 1a and 3b, the output sets of NC and PC LD phenotype embeddings from the ML LD embedding model 106b based on the UMAP algorithm are applied to the Sinkhorn algorithm for training and generating the ML distance model 106c as described with reference to FIG. 3b. The Wasserstein distance is optimized by iteratively performing a grid search over the set of parameters to find hyperparameters of the Sinkhorn algorithm that maximize the estimated Wasserstein distance between the set of NC LD phenotype embeddings and the set of PC LD phenotype embeddings. Then, the optimized hyperparameters output by the Sinkhorn algorithm may be used by the ML distance model 106c on other test sample LD embeddings to enable comparison of distances with the negative control LD embeddings.

The matrix distance plot in FIG. 4b is a mapping of the distance, when the toxicity prediction model 106 has been calibrated to using the viable input sample images from the assay plate 400 of FIG. 4a. The columns 0 to 9 and rows 0 to 9 of the matrix plot represent the distances between the NC LD embeddings associates with the viable images samples of the NC sample wells 402 of FIG. 4a. The columns 10-19 and rows 10-19 of the matrix plot represent the distances between the PC LD embeddings associated with the viable image samples of the PC sample wells 404 of FIG. 4a. It is evident that the DL toxicity model, i.e. the ML LD embedding model 106b and ML distance model 106c/d, has been trained as the distances between the NC LD embeddings have a dark gray shaded area 412 indicating a minimum distance therebetween. Similarly, for the PC LD embeddings which also have a dark grey shaded area 414 indicating a minimum distance therebetween. As well, the lighter grey shaded areas 416 indicate that the distance between the set of NC LD embeddings and the set of PC LD embeddings has been maximized (as much as possible) for this example. Given that the phenotypical distance is maximized in the areas 416 indicates that the corresponding PC samples have a toxic effect on the samples of the HepaRG cell-line. This is a clear indication that the DL toxicity model of the toxicity prediction system 106 has been calibrated and may be used to test the toxicity of a range of compounds in relation to HepaRG cell-line.

FIG. 4c illustrates another example distance matrix 420 of negative and positive control samples and also test samples for predicting toxicity of compounds of the test samples using the trained DL toxicity model 106b-106d of FIG. 4b of the toxicity prediction system 106. In addition to the viable NC and PC sample images output from the in-vitro micro assay represented by assay plate 400 of FIG. 4a, a plurality of viable images of test samples are also processed to predict the toxicity of the compounds applied to the test samples. In the matrix 420, cols 0 to 95 and 288 to 311 and rows 0 to 95 and 288-311 represent the viable images of the NC samples used from plate 400, cols 312 to 335 and rows 312-335 represent the viable images of the PC samples used from plate 400 to train the DL toxicity models 106b-106d of FIG. 4b, and cols 96 to 287 represent the viable images of the test samples used from plate 400 for testing. As can be seen, the dark grey area 422 in cols 0 to 95 and rows 0 to 95 of the distance matrix represents the distances between the NC LD embeddings, which have a minimum distance therebetween. There are some false positives giving a lighter shade of grey, which does not affect the performance of the toxicity prediction. Similarly, the dark grey area 424 in cols 312 to 335 and rows 312 to 335 of the distance matrix 420 represents the distances between the PC LD embedding, which also have a minimum distance therebetween. As well, the lighter grey shaded areas 426 of the distance matrix indicate that the distance between the set of NC LD embeddings and the set of test sample LD embeddings, which does not indicate a minimum distance from the NC LD embeddings but rather a larger distance indicating a higher toxicity effect on the HepaRG cell-line samples used in most, if not all, test samples.

FIG. 4d illustrates an example of a conventional toxicity prediction methodology used on a set of 14 compounds (e.g. compounds A, B, C, D, E, F, G, H, I, J, K, L, M, and N) applied to samples of a HepaRG cellular structure. In this example, the assay plate had 14 compounds, 8 doses, with 3 replicates for each dose. The 8 of the compounds were known to have a toxic effect on the HepaRG cellular structure and hence on the liver. These compounds were tested using commercial software that comes with the in-vitro microscopy hardware and conventional toxicity analysis. This conventional toxicity analysis is based on using the standard image analysis tools to analyse the data (e.g. stage 1), and standard toxicity characterisation based on vesicule count (e.g. stage 2) and analysis by researchers of dosage response (EC50) graphs (e.g. stage 3). In stage 4, only 8 compounds (e.g. compounds A, E, F, I, J, K, M and N) were found to have a toxic effect, but the conventional toxicity analysis methodology determined 6 of the toxic compounds (e.g. compounds B, C, D, G, H, and L) had no toxic effect. However, it is evident that the conventional toxicity analysis workflow does miss compounds having a toxic effect due to the 6 compounds they could not detect. When these compounds were applied to the toxicity prediction system 106 with the trained DL toxicity model 106b/106c as described with reference to FIGS. 4a-4c, all of the compounds were found to have a toxic effect. FIG. 4e is a schematic diagram illustrating an example of the conventional toxicity prediction results for compounds A, B, C, D, E, F, G, and I using the conventional approach to predicting toxic compounds. The bile vesicules count and cell count for the positive control compound, i.e. Chlorpromazine, is indicated within the dashed box and the bile vesicules count and cell count for the negative control compound, i.e. DMSO, is indicated by the solid box. As can be seen, it is very difficult to predict whether compounds B, C, D, and G have a toxic effect when considering only bile vesicules count and cell count. FIG. 4f is a schematic diagram illustrating an example of the toxicity prediction results of the trained DL toxicity model for compounds A, B, C, D, E, F, G, and I using the trained DL toxicity model 106b/106c. The schematic diagram of FIG. 4f is a graphical representation of novel phenotypical distance metric (y-axis) of the deep learning based phenotypes extracted using a cell-based imaging classification backbone (e.g., Resnet50). The phenotypical distance metric for the positive control compound, i.e. Chlorpromazine (dashed box), and the negative control compound, i.e. DMSO (in solid box) are illustrated. The phenotypical distance metric compares every compound to the negative control compound (average phenotypical representation) hence the larger distances (y-axis) between the tested compounds (e.g. compounds A, B, C, D, E, F, G, and I) and the average negative DMSO phenotypical profile. As can be seen, the distance between the compound phenotypes and the reference negative profile (average DMSO) is for each tested compound higher than the 75^{th} percentile of the DMSO distribution. This difference in addition to similarity to the positive control compound (Chlorpromazine) can qualify the phenotypical effects as toxic (difference from DMSO alone does not qualify toxicity alone). As can be seen, compounds A, B, C, D, E, F, G, and I are more readily able to be predicted to be toxic using the trained DL toxicity model 106b/106c, the results of which can be used to more effectively predict the toxicity of compounds compared with conventional toxicity prediction methodologies.

FIG. 5a illustrates an example of a quality control process 500 for use in image analysis and feature extraction system 104 of toxicity pipeline 100 of FIG. 1a for automatically identifying viable analysable images of images of samples 103 captured from the HTS in-vitro microscopy system 102 of FIG. 1a. The QC process 500 is used to determine which samples or groups of samples from the set of wells on an assay plate are analysable and not analysable. The QC process 500 used ML techniques to assist in the determination of which images captures of the wells may be retained or discarded for use in downstream analysis. The set of viable images are analysable in that the cellular structures are sufficiently well defined for downstream image analysis processing such as, without limitation, for example extracting phenotype feature representations from the cellular structures of the images for use in training and/or input to the DL/ML models 106b-106c of the toxicity prediction system 106 as described with reference to FIGS. 1a to 4e. For example, the set of viable images 105 output from QC process 500 improves the robustness and reliability of the trained DL models 106a-106c, which further enhances the robustness and accuracy of the toxicity predictions output by the toxicity prediction system 106 in relation to test samples with compounds applied thereto for toxicity prediction.

Although the set of viable images 105 output by the QC process 500 is described with reference to toxicity prediction system 106, this is by way of example only, it is to be appreciated by the skilled person that the set of viable images 105 output by the QC process 500 may be input to any other downstream process associated with analysing, classifying, and/or predicting/estimating one or more aspects or properties of said images depending on the type of HTS in-vitro microscopy assay that is performed. For example, an HTS in-vitro microscopy assay may be used in a drug/compound research programme for use in determining properties or effects of one or more compounds on cellular structure samples of the in-vitro microscopy assay such as, without limitation, toxicity of compounds applied to cellular structure samples, efficacy of a compound's effect on cellular structure samples, and/or any other type of property of a compound when applied to a cellular structure sample in an in-vitro microscopy assay and the like.

In this example, the QC process 500 identifies a first set of viable images of samples from the images 103 captured from the HTS in-vitro microscopy system 102 and pre-processes the first set of viable images into a final set of viable images 105 for input to, without limitation, for example toxicity prediction system 106 and/or any other compound analysis system or downstream workflow process/analysis system. With reference to the toxicity prediction system 106, QC process 500 outputs a set of viable images of samples 105 for use in training the DL toxicity models 106a/106b/106c of the toxicity prediction system 106 of FIG. 1a and/or as input to the trained DL models 106a/106b/106c of the toxicity prediction system 106 as described with reference to FIGS. 1a to 4e. The toxicity prediction system 106 receives the set of viable images associated with the plurality of samples in which at least one group of the plurality of samples have compounds applied thereto for testing, and/or as a positive control group for training said DL models 106b-106c of the toxicity prediction system 106.

On receiving a set of images captured from a plurality of samples of an assay plate (e.g. assay plate 102c, 300 or 400 of FIG. 1a, 3a or 4a), the QC process 500 identifies viable samples of cellular structures from an in-vitro microscopy assay for further downstream analysis. The QC process 500 includes the following steps of:

In step 502, automatically identifying a first set of images of samples useful for analysis from the received set of images of samples captured from the plurality of samples of an assay plate. For example, the received set of images of samples are automatically analysed to determine whether features of the spheroid/cellular structure of the samples exist and to determine whether these features highlight possible artifacts on the image, or out of focus areas and/or other imaging defects. Artifacts can bias the prediction in relation to the downstream analysis using ML model(s) and the like. For example, toxicity prediction system 106 uses received images of samples 105 to predict whether a compound is toxic or non-toxic, so any artifacts can be detrimental to the trained and/or prediction of toxicity as they may mask the effects of the compound applied to the cell structure. For example, these artifacts may mask the dose at which compound is actually toxic (e.g. doses having 50% toxic effects).

The automatic identification uses one or more ML models to estimate and predict whether the sample of each well is analysable or not. For example, automatically identifying a first set of images of samples may include inputting the received set of images of samples from the assay to one or more ML model(s) configured for identifying one or more regions of each image in the set likely to have cellular structures therein. The set of images with the identified regions may be input to one or more further ML model(s) for identifying whether the cellular structures within the identified regions of each image are suitable for further downstream analysis or not. Those images from the received set of images with regions containing cellular structures that are determined to be analysable are selected to form the first set of images of samples that are viable for further downstream analysis.

In step 504, for each image of a sample in the first set of images of samples, generating a set of 2-dimensional (2D) images for the sample, where the set of 2D images for said sample includes capturing multiple 2D image slices for the sample in the assay plate in which the multiple 2D image slices are taken along a z-axis of said sample. The set of 2D images for each sample in the first set of images of the samples are taken at different z-axis locations such that a 3D representation of said each sample if formed. The multiple 2D images for each sample forms a 3D representation of each well of said each sample, which may be further processed and represented as a fused/compressed 2D representation.

In step 506, identifying from the sets of 2D image slices a set of viable samples. For example, for each sample in the first set of images of samples, the set of 2D image slices may be fused, compressed or combined to form a 2D representation of the 3D structure of the sample, where the 2D representation enhances the cellular structures within the sample. The cellular structures of each fused 2D representation may be further analysed to identify whether they are sufficiently distinguishable or well defined for use in downstream analysis (e.g. for input to toxicity prediction system 106). For example, a fused 2D representation is sufficiently distinguishable or well defined when the quality of the images is determined to be appropriate for extracting one or more metrics or phenotypical representations of cellular structures from the images. Those samples from the first set of images of samples with sets of 2D image slices that are identified to be sufficiently distinguishable or appropriate for further analysis form the set of viable samples for analysis.

In step 508, outputting data representative of said set of viable samples for analysis as a set of images 105. For example, the each fused 2D representation image of the viable set of samples may form the set of images 105 of FIG. 1a that may be input to downstream processes for analysis, such as toxicity prediction system 106.

FIG. 5b illustrates an example of a first quality control (QC) process 510 for use in step 502 of QC process 500 of FIG. 5a for selecting a first set of viable images of samples suitable for further downstream analysis. The first QC process 510 is configured to automatically identify the first set of images of samples that are viable for analysis, e.g. for input to toxicity prediction system 106 as described with reference to FIGS. 1a to 5a. The first QC process 510 may include the steps of: In step 512, pre-processing each image of a sample from the received set of images of samples 103 from the HTS in-vitro assay system 102 to form a pre-processed set of images of said samples. The pre-processing may include performing image analysis and/or processing based on, without limitation, for example correcting illumination, lighting, illumination field correction, artifact reduction/interpolation or any other image processing algorithm for processing an image to further enhance the cellular structures that may be contained within said image. In step 514, inputting said pre-processed set of images of said samples to a machine learning (ML) region of interest (ROI) model trained and configured for identifying, for each input image of a sample, one or more regions of interest in the input image of the sample, where the region of interest includes a cellular structure of the corresponding sample. The ML region of interest (ROI) model may be based on any type of neural network (NN) structure such as, without limitation, for example a feed forward NN (FNN), recursive NN (RNN), artificial NN (ANN), convolutional NN (CNN), any other type of NN suitable for identifying regions of interest in an image associated with cellular structures, modifications thereto, combinations thereof. For example, the ML ROI model may be based on a CNN trained to identify ROI associated with cellular structures within an input image and/or output images focussed on an identified ROI associated with cellular structures within an input image. The ML ROI model may be trained using a CNN structure and a labelled or annotated training image data set, where each image in the training image data set is labelled or annotated with information associated with whether a cellular structure is present, and if a cellular structure is present, the region of interest within the image.

At this stage, those images in the input set of images of samples in which the ML ROI model does not detect a region of interest may be discarded from the set of images of samples, as these as more likely not able to be analysed. Alternatively, for each image that the ML ROI model does not detect a region of interest, then the region of interest may default to the entire input image for further analysis in step 516. That is, the region of interest may be identified as the entire input image, which may be input to step 516.

In step 516, inputting each of the images with an identified region of interest containing the cellular structure of the sample to a ML image viability model trained and configured for classifying whether said cellular structure in said input image is analysable. Thus, each input image may be classified with a label or probability value representative of the viability of the input image to be analysable. Should the label or probability value indicate that the input image is analysable (e.g. the probability value may be greater than or equal to a predetermined viability probability threshold, or the label indicates the image is viable), then the input image is placed into the first set of images of samples, which are considered viable. The remaining input images are discarded with a label indicating the image is not viable, or with a probability value less than the predetermined viability probability threshold.

The image viability model may be based on, without limitation, for example any ML algorithm or technique that may be used to classify whether the cellular structure within said ROI in said image is analysable or not. This may be based on identifying, from each input image, whether any phenotypical features associated with cellular structures (e.g. spheroid, nuclei, vesicules and the like) within the input image are present and assessing whether enough phenotypical features are present in the cellular structure of the sample captured in the image will enable further analysis of the cellular structure of the sample within the image. For example, the ML algorithm used to train the image viability model may be based on, without limitation, for example support vector machines (SVMs) or NN classifiers/structures and the like. As an example, the image viability model may be based on a set of SVMs each associated with classifying a particular phenotypical feature, or classifying a cluster of phenotypical features, of the cellular structure of the sample captured within an input image, where each SVM outputs a positive or negative classification indicating whether the particular phenotypical feature is present. If enough SVMs output a positive classification indicating the corresponding particular phenotypical features are present, then the input image may be considered to be an image that is viable for analysis.

In step 518, outputting the first set of images of samples including data representative of those images of samples that are classified to be analysable in step 516. The output first set of images of samples may be further processed such as, without limitation, for example in steps 504-508 of QC process 500, to further enhance the cellular structures contained in each image and/or for further selection in relation to viability of said first set of images of the samples. Alternatively or as an option, the first set of images of samples that are considered viable that are output from first QC process 510 may be output from the image analysis system 104 as a set of images 105 for input to the toxicity prediction system 106 or other downstream process for analysis and the like.

FIG. 5c illustrates an example of an image analyser 520 for implementing steps 512 and 514 of first QC process 510 of FIG. 5b in which each image of a sample is pre-processed and the regions of interest containing cellular structures within each image of a sample are identified. In this example, each image in the set of images 103 of samples output from the HTS in-vitro microscopy assay system 102 is pre-processed by image pre-processing unit 522. In this example, each input image 522a to the image pre-processing unit 522 has an illumination function 522b applied thereto and the resulting illumination corrected image 522c output from the first image pre-processing unit 522 is passed towards the image ROI unit 524. The illumination function 522b may be configured to correct for inhomogeneous florescence illumination by the image capturing device in the HTS in-vitro microscopy system 102. Although image pre-processing unit 522 performs illumination field correction on each image, this is by way of example only and the specification is not so limited, it is to be appreciated by the skilled person that image pre-processing unit 522 may perform one or more other image processing functions to such as image focusing, illumination field correction, sharpening, saturation, artifact reduction, correction and/or interpolation, any other image processing algorithm, combinations thereof, modifications thereto and the like, in which the cellular structure of the sample captured each input image is further enhanced for use by image ROI unit 524.

Each of the pre-processed set of images of said samples output from the image pre-processing unit 522 are input to the image ROI unit 524. The image ROI unit 524 is configured to include a ML region of interest (ROI) model based on an CNN architecture (e.g. CNN-based (CellPose (RTM)) segmentation) that is trained and configured for identifying each segment of an image and determine which segments contain a cellular structure, where the segments containing a cellular structure form the ROI of the image. CNN-based segmentation (e.g. CellPose) architectures may be used on microscopy images for segmentation of cell bodies, membranes and nuclei. For example, CellPose is a deep learning CNN architecture that has been trained on a dataset of highly varied images of cells, containing over 70,000 segmented objects. These may be retrained and/or fine-tuned on a training dataset of images focussed on the cellular structures of the microscopy assay images that are expected to be used in the downstream analysis. Although the CNN-based Cell Pose segmentation architecture was described, this is by way of example only and the specification is not so limited, it is to be appreciated by the skilled person that any other suitable ML algorithm/model may be used that performs identification of ROI containing cellular structures in relation to the input images of the samples. The ML ROI model may be trained using the CNN structure using labelled training dataset including a plurality of images, each of the images annotated with a label or annotation comprising data representative of whether a cellular region of interest is present, and/or the location of the region of interest within the image etc. For example, the training image dataset may include images that are labelled or annotated with information associated with whether a cellular structure (e.g. phenotypical features of cellular structures and/or macro cellular structures, cell spheroids, cell nuclei, cell mutations, perturbations, cell cholestasis, relevant biological features and the like) is present, and if a cellular structure, a perturbed cellular structure or the remains of a cellular structure is present, the region of interest containing the cellular structure within the image. Thus, the ML ROI model is trained and configured to identify/predict the segments/ROI of the input image containing the remaining/mutated/perturbed cellular structure in the image of the sample, and output this information for use in downstream processing.

For example, an input image of a sample 524a may contain a cellular structure such as a cell spheroid object containing cell nuclei, where only those segments of the image containing the cell nuclei are identified and form the region of interest of the image. In another example, an input image of a sample 524b may contain a cell spheroid object representing a cellular structure undergoing cholestasis, i.e. the cellular structure has been perturbed/mutated by a compound and, without limitation, for example nuclei may be disappearing and the like. In such as case, the ML ROI model has been trained to identify the segments/ROI of the image containing the remaining/mutated/perturbed cellular structure in the image of the sample. The image ROI unit 524 may be configured to output only those parts of the image 524a containing the ROI. Alternatively or additionally, the image ROI unit 524 may be configured to the input image 524a annotated with data representative of the ROI for use by other processing units/downstream processing in focussing on the cellular structure contained in the ROI of the image. In any event, the image ROI unit 524 outputs a set of image data representative of each of the set of pre-processed input images of the samples and, for each image in the set, the ROI containing the cellular structure of the sample within said each input image. The output image and ROI data of the image analyzer 520 may be used in steps 516 and 518 of first QC process 500, which may be implemented as an image viability system configured for extracting the features in the ROI of each image of a sample and determine whether said each image is of good quality of bad quality, i.e. is viable or not viable for further analysis, or each image is analysable or not analysable. The output of the image viability system will comprise a set of images of the samples that are analysable/viable and may form the set of images 105 that can be input to further downstream processes such as, without limitation, for example toxicity prediction system 106.

FIG. 5d illustrates an example of an image viability system 530 as part of a QC pipeline for implementing steps 516 and 518 of first QC process 510 of FIG. 5b for use in classifying whether the cellular structure in the ROI of each input image of a sample is analysable or not (e.g. viable for further downstream analysis/processing). The image viability system 530 may include the image analyser 520 of FIG. 5c, which receives the set of microscopy images of samples 103 output from the HTS microscopy assay system 102 of FIG. 1a, and outputs for each image in the set of images 103 data representative of each image and an ROI containing features of cellular structures for each image. The set of images and corresponding ROIs are input to the phenotype sampler 532, which, for each image in the set of images identifies and/or classifies whether the ROI of said each image has n phenotypical features (e.g. P1, P2, P3, . . . , Pn). Clustering is performed for each of the n phenotypical features detected within the ROI of each image of a sample. Each of the n phenotypical features has a one class SVM 536a-536n, trained for classifying the ROI of the image against one of the n phenotypical feature clusters. Each of the SVMs 536a-536n outputs a classification of whether the ROI of the image has the corresponding phenotypical feature P1, P2, . . . . Pn, respectively. Each cluster of the n phenotypical features P1, P2, P3, . . . , Pn is input to the corresponding SVM 536a-536n, each of which outputs a probability or likelihood that the corresponding phenotypical feature is within the ROI of said image of the sample. These n output probabilities are combined to provide an output classification value 538 indicating whether the image is analysable or not (e.g. viable or not). Those images in which the classification value 538 indicates the image to be analysable or viable (e.g. the classification value 538 is greater than a viability threshold value) is selected to form the first set of images of the samples that are viable.

FIG. 5e illustrates an example of a second quality control (QC) process 540 for use in steps 504-508 of QC process 500 enhancing and selecting a final set of viable images of samples for downstream analysis such as, without limitation, for example an input set of images 105 to the toxicity prediction system 106 of FIG. 1a. Once the first set of images of the samples that are viable have been output from step 502 of QC process 500 of FIG. 5a or from the image viability system 530 of FIG. 5c, then step 504 of QC process 500 may be performed where, for each image of a sample from the first set of viable images, a 3D representation of each well sample corresponding to said each image of the sample is captured by the microscopy imaging device. The 3D representation for each image of a sample is generated by the microscopy imaging device (e.g. ImageXpress® capturing a set of 2-dimensional (2D) images for the sample, where the set of 2D images for said sample includes capturing multiple 2D image slices of the sample in the well sample in the assay plate. The multiple 2D image slices are taken at different z-focuses or at different focuses along a longitudinal z-axis of said sample well. The set of 2D images for each image of a sample in the first set of viable images are taken at different z-axis locations such that a 3D representation of said each sample is formed. The set of 2D images for each sample forms a 3D representation of each well that corresponds to an image of the sample from the set of viable images. Each set of 2D images for each sample is enhanced to emphasize the cellular structure in each 2D image by fusing or compressing/combining the set of 2D images into a single 2D representation of said image of the sample. The second QC process 540 for enhancing and selecting a final set of viable images of samples for downstream analysis includes the following steps of:

In step 542, identifying, for each set of 2D images of a sample from the set of viable images of samples, foreground, background and multiple uncertain feature areas or classes of the cellular structure in each of the 2D image slices from said set of 2D images. The multiple uncertain feature areas or classes include multiple uncertain foreground feature areas or classes and multiple uncertain background feature areas or classes. For example, for each image in a set of 2D images, separate every x/y pixel value into multiple classes including, without limitation, for example a foreground class, a background class, and multiple uncertain foreground classes and multiple uncertain background classes.

In step 544, for each set of 2D images of a sample, iteratively combining the identified foreground, background and multiple uncertain feature areas or classes of the 2D image slices in the set of 2D images of a sample to generate a single 2D image of the cellular structure of said sample. For example, the iterative process takes each foreground class and mixes it with uncertain foreground class to try and optimize the projection between the two classes in an iterative process by smoothing this projection in a local area. The iterative optimization process may have two criteria, the final projection needs to be locally smooth in terms of z-projection in a local area around the foreground class and the local intensity in the local area is maximum possible intensity. The iterative combining may be based on a ML smoothing model that iteratively smooths the foreground classes and uncertain foreground classes.

In step 546, for each single 2D image of a sample, selecting the iteratively generated single 2D image of the sample for the final viable image sample set based on the quality of the single 2D image of the sample. For example, the quality of the single 2D image sample may be assessed based on whether there are any sharp deviations between neighbouring foreground feature areas and neighbouring uncertain foreground areas and the like, or any sharp deviations between neighbouring background feature areas and neighbouring uncertain background areas and the like. For example, if there are too many “jumps” between neighbouring foreground areas/classes and neighbouring uncertain foreground areas/classes at the end of the iterative procedure, or the resulting single 2D image still does not satisfy the criterion in relation to image intensity for a local area of a z-projection, or if the iterative procedure did not converge to within the appropriate error threshold, then the single 2D image is not viable and may be selected for discarding. The remaining single 2D images of each sample may be used to form the final set of viable images.

In step 548, outputting the final set of viable images comprising image data representative of the single 2D images of each sample selected in step 546. The final set of viable images may form the set of images 105 that are input to downstream processes.

FIG. 5f illustrates another example of a smooth manifold extraction (SME) system 550 for implementing steps 504-508 of QC process 500 of FIG. 5a and/or steps 542-548 of second QC process 540 of FIG. 5e for use in further enhancing and selecting a final set of viable images 567a-567p for output from the image analyser system 104. The SME system 550 includes chain of processing units including a 3D image representation unit 552, profiling unit 554, FFT unit 556, pixel classification unit 558-560 for foreground/background classification/clustering, iterative SME unit 562, and final image selection unit 564, and output unit 566. The output unit 566 outputs the final set of viable images 567a-567p, which may be used as input as the set of images 105 for further downstream analysis processes such as, without limitation, for example input to toxicity prediction system 106.

The 3D image representation unit 552 is configured to perform step 504 of FIG. 5a, which receives the first set of viable images of samples that have been output from step 502 of QC process 500 of FIG. 5a or from the image viability system 530 of FIG. 5c. The 3D image representation unit 552 captures a 3D representation of the sample in each sample well 551 corresponding to the sample of each viable image of the sample. For example, a microscopy imaging device 553 (e.g. ImageXpress® is directed to capture a set of 2-dimensional (2D) images 555 corresponding to the sample well 551 of a viable image of the sample. The set of 2D images 555 for the sample includes a plurality of 2D image slices 555a-555k of the sample from the corresponding sample well 551 of the assay plate. The sample well 551 has a longitudinal z-axis 551a, an x-axis 551b and y-axis 551c, where the x-axis 551b and y-axis 551c are orthogonal to each other and also orthogonal to the longitudinal z-axis. The 2D image slices 555a-555k are taken at different z-focuses or at different focuses along a longitudinal z-axis 551a of said sample well 551. The set of 2D images 555 for each viable image of the sample are taken at different z-axis locations such that a 3D representation of said each sample is formed. The set of 2D images 555 for each sample forms a 3D representation of each well 551 that corresponds the viable image of said each sample from the received set of viable images.

The remaining processing units 554-566 of the SME system 550 are configured to process each set of 2D images 555 for each sample to enhance/emphasize the cellular structure of the sample represented in each 2D image. This is performed by fusing or compressing/combining each set of 2D images 555 of each sample into a single 2D representation 565 of said sample. In essence, these remaining processing units 554-566 are configured, for each sample, to find the z-focus for each pixel of each set of 2D images 555 that corresponds to the relevant structure of cells/cellular structure for said each pixel.

In profiling unit 554, the profile of each 2D image slice 555a in the set of 2D images 555 is extracted in which any (x,y) position corresponds to a profile of focus values in the z direction 551 of the sample well 551, which is made of direct intensity values in case of confocal image or the SML values for wide-field epifluorescence images. Each of the 2D image slices 555a-555k (e.g. profiles) that pass through some foreground signal contain lower-frequency components. The profiles of each of the 2D image slices 55a-55k are passed to the FFT unit 556. The FFT unit 556 performs, for each set of 2D images of the sample, a Fast Fourier Transform (FFT) on each of the profiles of the 2D image slices within the set of 2D images 555 of the sample to determine the power frequency spectrum of each profile of the 2D image slices 555a-555k. The FFT profiles of the 2D image slices 555a-555k are passed to the pixel classification unit 558-560 for foreground/background classification and clustering.

The pixel classification units 558-560 uses a ML classification algorithm/model to perform a multi-class classification on the FFT profiles of the 2D image slices 555a-555k to classify each (x,y) position or pixel of each profile of a 2D image slice into one of a plurality of labels associated with a foreground class 559a, a background class 559b and/or multiple uncertain foreground/background classes 559c-5591. The ML classification algorithm may be based on, without limitation, for example an multi-class k-means algorithm, a multi-class UMAP algorithm, and/or any other suitable multi-class classification algorithm or model capable of classifying the (x,y) position of each pixel of each of the FFT profiles of the 2D image slices 555a-555k of the set of 2D images 555 into a label associated with a foreground class 559a, a background class 559b or multiple uncertain foreground/background classes 559c-559l. The pixel classification unit 556 outputs to the SME unit 562 a labelled/annotated FFT profile 561 representing the 2D image slices 555a-555k in which each (x,y) pixel of each 2D image slice has been labelled with a classification associated with a foreground class 559a, a background class 559b or one of the multiple uncertain foreground/background classes 559c-559l.

The SME unit 562 is based on an ML SME model/algorithm 563 (or iterative SME optimisation process) that iteratively processes data representative of the set of 2D images for each sample based on using the determined foreground, background, and uncertain foreground and background classes assigned to pixels of the 2D image slices of the set of 2D images for each sample. The ML SME model 563 uses an iterative SME algorithm. The ML SME model 563 is configured perform an iterative SME optimisation procedure that uses a cost function that balances local smoothness and proximity to the maximum of focus value is minimized for combining data representative of the 2D image slices for said sample to obtain the final smoothed index map 563m of said sample. Note that the first index map 563a is highly discontinuous at the beginning of the iterative SME optimisation procedure, but should be smoothed as the iterations of the SME optimisation procedure converge to the final smoothed index map 563m while preserving fine detail on the foreground. The final smoothed index map 565m is sent to the extraction and selection unit 564.

For example, the SME algorithm 563 of the SME unit 562 looks at the foreground pixels mixed with uncertain foreground classes, and attempts to optimize the projections of these types of foreground classes of the 2D image slices 555a-555k and fuse/compress them into a final single 2D image 565a (or final index map 563m) using the iterative SME optimisation procedure. The iterative SME optimisation procedure smooths this projection in each iteration from the first iteration (e.g. iteration 001) to a final iteration (e.g. iteration 571) when a final error threshold has been reached/met and/or a maximum number of iterations has been met/reached. The iterative SME optimisation procedure has two criteria to meet, the first criteria is that the final projection (e.g. final index map 563m or final single 2D image) needs to be locally smooth in terms of z-projection and the second criteria is that the local intensity is maximum possible intensity (e.g. using a Gaussian energy function). These criteria is used in each iteration from the first iteration to the final iteration as the 2D image slices are fused/compressed together to form a final single 2D image. Once the iterative SME optimisation procedure of the SME algorithm 563 has converged to a final index map 563m and/or the maximum number of iterations has been met, then the resulting final index map 563m is used to form the final single 2D image 565a. The resulting final index map 563m or the final single 2D image 565a is passed to the extraction and selection unit 564 for determining whether the final single 2D image 565a should be included in the final set of viable images.

In extraction and selection unit 564, the final smoothed index map 563m output from the iterative SME algorithm 563 is analysed and a plurality of voxels corresponding to this index map 563m are extracted from the original stack to produce the final single 2D image 565a. The extraction and selection unit 564 using selection unit 565b selects whether to include the final single 2D image 565a in the final set of viable images of samples for sending to output unit 566. The selection unit 565b determines whether the final single 2D image 565a or the index map 563m corresponding to the final single 2D image 565a is of sufficient quality or sufficient smoothness for output from extraction and selection unit 564. For example, the selection unit 565b determines whether two criteria have been met by the iterative SME algorithm 563 or final single 2D image 565a, the first criteria is based on determining whether the projection of the final single 2D image 565a or final index map 563m of the iterative SME algorithm 563 is locally smooth in terms of z-projection and the second criteria is based on determining whether the local intensity is at a maximum possible intensity.

For example, the first criteria based on examining the smoothness of each local area of pixels of the final single 2D image 565a (or final index map 563m). In this case, each local area of pixels of the final single 2D image 565a (or final index map 563m) such as, without limitation, for example each neighbourhood of 5×5 pixels in the final 2D image 565a (or final index map 563m), the z-level in said each local area (e.g. neighbourhood of 5×5 pixels) should be smooth in terms of z-level, where there should not be any deviations or “jumps” in intensity (e.g. not go from an intensity level of 1 directly to 17) when move from 1 pixel to another in the local area. The second criteria is based on the intensity of the z-projection of each local area of pixels of the final single 2D image 565a (or final index map 563m). This second criteria is met when, within each local area (e.g. neighbourhood of 5×5 pixels), the z projection taken on the local area (e.g. neighbourhood of 5×5 pixels) has an intensity value based on the local area (e.g. neighbourhood of 5×5 pixels) that is at a maximum z intensity. In the selection unit 565b, if these two main criteria are not met, then the selection unit 565b discards the final single 2D image 565a (or final index map 563m), which does not form part of the final set of viable images 567. Otherwise, the final single 2D image 565a of the sample (or final index map 563m) is output by the extraction and selection unit 564 for sending to the output unit 566 and the output final single 2D image 565a is added as a final viable image 567a of the final set of viable images 567.

Although the above first and second criteria are described herein, this is by way of example only, the person skilled in the art would appreciate that modifications to these two criteria and/or other criteria may be applied for use in selecting/discarding the final single 2D image 565a when sending to the output unit 566. For example, these modifications and/or other criteria may be based on, without limitation, for example the first criteria may further modified to count the number of “jumps” of deviations in intensity for use in determining whether to discard/select the final single 2D image 565a (e.g. the final single 2D image 565a may be selected for output if the number of “jumps” or deviations in intensity is below a minimum “jump” threshold value/count, otherwise it is discarded), and/or the selection unit 565b may further take into account whether the iterative SME algorithm converged within an pre-determined error threshold (e.g. the final single 2D image 565a may be selected for output if the convergence error of the iterative SME algorithm less than or equal to the pre-determined error threshold value, otherwise it is discarded), and/or any other criteria for assessing whether the final single 2D image 565a of each sample is of sufficient quality and/or analysable by further downstream processes and the like.

The output unit 566 stores each of the output final single 2D images 565a that are selected for output by extraction/selection unit 564 as a plurality of images that forms the final set of viable images of the samples. The output unit 566 may then send the final set of viable images of the samples towards further downstream processes such as, without limitation, for example as an input set of images 105 to the toxicity prediction system 106.

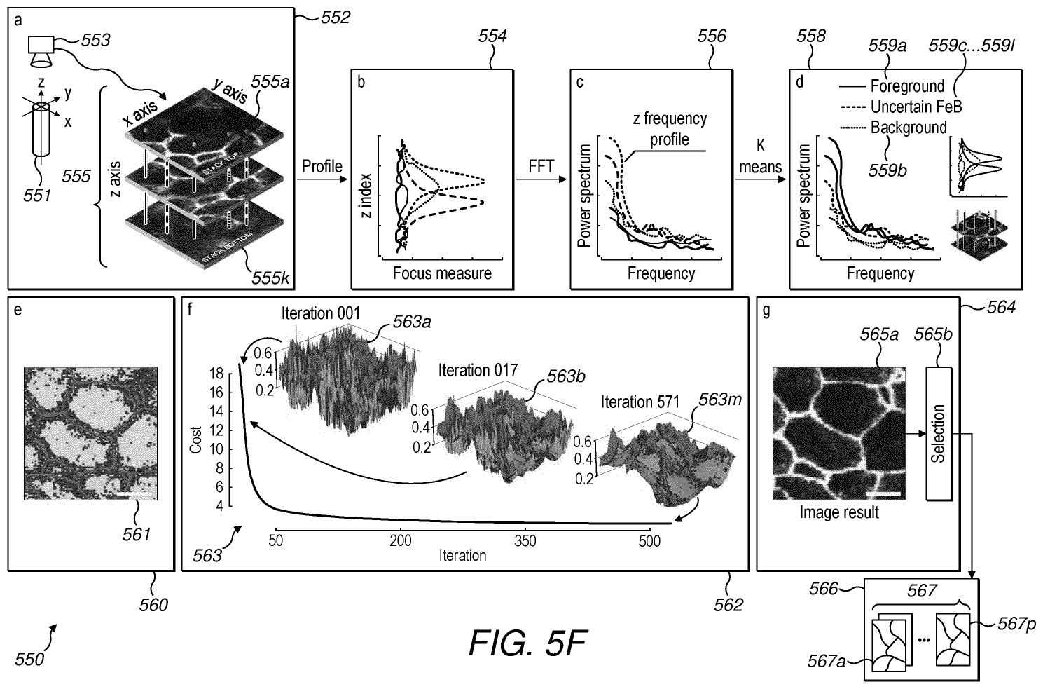

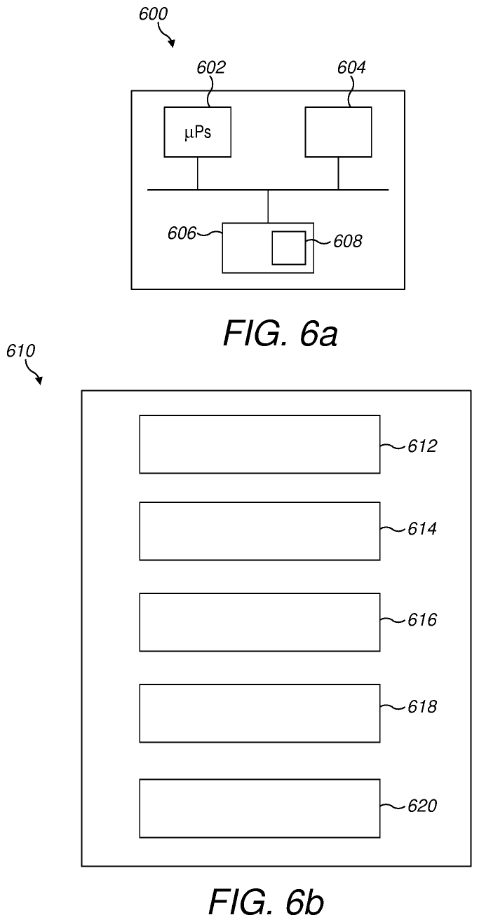

FIG. 6a is a schematic illustration of a system/apparatus for performing methods described herein. The system/apparatus shown is an example of a computing device. It will be appreciated by the skilled person that other types of computing devices/systems may alternatively be used to implement the methods described herein, such as a distributed computing system.

The apparatus (or system) 600 comprises one or more processors 602. The one or more processors control operation of other components of the system/apparatus 600. The one or more processors 602 may, for example, comprise a general purpose processor. The one or more processors 602 may be a single core device or a multiple core device. The one or more processors 602 may comprise a central processing unit (CPU) or a graphical processing unit (GPU). Alternatively, the one or more processors 602 may comprise specialised processing hardware, for instance a RISC processor or programmable hardware with embedded firmware. Multiple processors may be included.

The system/apparatus comprises a working or volatile memory 604. The one or more processors may access the volatile memory 604 in order to process data and may control the storage of data in memory. The volatile memory 604 may comprise RAM of any type, for example Static RAM (SRAM), Dynamic RAM (DRAM), or it may comprise Flash memory, such as an SD-Card.

The system/apparatus comprises a non-volatile memory 606. The non-volatile memory 606 stores a set of operation instructions 608 for controlling the operation of the processors 602 in the form of computer readable instructions. The non-volatile memory 606 may be a memory of any kind such as a Read Only Memory (ROM), a Flash memory or a magnetic drive memory.

The one or more processors 602 are configured to execute operating instructions 608 to cause the system/apparatus to perform any of the methods described herein. The operating instructions 608 may comprise code (i.e. drivers) relating to the hardware components of the system/apparatus 600, as well as code relating to the basic operation of the system/apparatus 600. Generally speaking, the one or more processors 602 execute one or more instructions of the operating instructions 608, which are stored permanently or semi-permanently in the non-volatile memory 606, using the volatile memory 604 to temporarily store data generated during execution of said operating instructions 608.

FIG. 6b is a schematic illustration of a system 610 for performing methods described herein. The system 610 shown is an example of a computing device, system and/or cloud computing system and the like. It will be appreciated by the skilled person that other types of computing devices/systems may alternatively be used to implement the methods described herein, such as a distributed computing system. The system 610 includes a receiver module/unit 612, a first ML model module/unit 614, a second ML model module/unit 616, a distance comparison module/unit 618 and an output module/unit 620 that may be connected together or communicate with each other as necessary for implementing the methods and/or apparatus/system as described herein.

For example, the receiver module 612 may be configured for receiving a set of images associated with the plurality of samples. The first ML model module 614 may be configured for inputting each image of the set of images to a first ML model 106a configured for predicting phenotype features 107 of the cellular structure within the sample associated with said each image. The second ML model module 616 may be configured for inputting each of the predicted phenotype features 107 associated with each sample to a second ML model 106b configured for predicting a lower dimensional phenotype feature embedding 108 of said each sample. The distance comparison module 618 may be configured for comparing the distance between lower dimensional phenotype feature embedding 108 of said each sample that of a sample with compound having a known toxicity, which may output data representative of said comparison 109. The output module 620 may be configured for outputting, for each sample, an indication of the toxicity of said each sample and applied compound thereto based on said comparison 109.

Implementations of the methods described herein may be realised as in digital electronic circuitry, integrated circuitry, specially designed ASICs (application specific integrated circuits), computer hardware, firmware, software, and/or combinations thereof. These may include computer program products (such as software stored on e.g. magnetic discs, optical disks, memory, Programmable Logic Devices) comprising computer readable instructions that, when executed by a computer, such as that described in relation to FIG. 6a or 6b, cause the computer to perform one or more of the methods described herein.

Any system feature as described herein may also be provided as a method feature, and vice versa. As used herein, means plus function features may be expressed alternatively in terms of their corresponding structure. In particular, method aspects may be applied to system aspects, and vice versa.

Furthermore, any, some and/or all features in one aspect can be applied to any, some and/or all features in any other aspect, in any appropriate combination. It should also be appreciated that particular combinations of the various features described and defined in any aspects of the specification can be implemented and/or supplied and/or used independently.

Although several embodiments have been shown and described, it would be appreciated by those skilled in the art that changes may be made in these embodiments without departing from the principles of this disclosure, the scope of which is defined in the claims.

1-15. (canceled)

16. A computer-implemented method for predicting toxicity of one or more compounds applied to a plurality of samples of a cellular structure in an in-vitro microscopy assay, the method comprising:
receiving a set of images associated with the plurality of samples;
inputting each image of the set of images to a first ML model configured for predicting phenotype features of the cellular structure within the sample associated with said each image;
inputting each of the predicted phenotype features associated with each sample to a second ML model configured for predicting a lower dimensional phenotype feature embedding of said each sample;
comparing the distance between the lower dimensional phenotype feature embedding of said each sample with that of a sample applied with a compound having a known toxicity; and
outputting, for each sample, an indication of the toxicity of said each sample and applied compound thereto based on said comparison.

17. The computer-implemented method of claim 16, wherein the first ML model is a neural network or convolutional neural network, CNN, model, the neural network or CNN model is trained for classification using cellular image training data and the predicted phenotype features are embedded within a full layer of the trained neural network or CNN model, the method comprising outputting the phenotype feature embeddings from the full layer.

18. The computer-implemented method claim 16, wherein the second ML model is based on a Uniform Manifold Approximation and Projection, UMAP, algorithm or t-SNE algorithm for dimensional reduction of the phenotype feature embedding of a sample, wherein the phenotype feature embedding is mapped to a lower dimensional vector space for use in comparing the distance between the phenotype feature embedding and that of a sample with a compound having a known toxicity.

19. The computer-implemented method of claim 18, further comprising:
training the second ML model on the UMAP technique using unsupervised training based on negative and positive control samples of the plurality of samples for predicting a toxicity distance metric associated with the samples with compounds applied thereto having a known toxicity; or
training the second ML model comprises iteratively performing a grid search over a set of hyperparameters of the UMAP technique for selecting those hyperparameters that maximise the differences between the negative control samples and the positive control samples.

20. The computer-implemented method of claim 18, wherein indicating the toxicity of the phenotype feature embedding of a sample with compound applied thereto comprises:
applying the phenotype feature embedding of a sample with compound applied thereto to the second ML model for outputting a lower dimensional embedding of said sample with compound applied thereto; and
determining (325, 325a, 325b, 326) an indication of the toxicity of said sample with compound applied thereto based on comparing the distance between said lower dimensional embedding and the embeddings of one or more samples with compounds applied thereto having known toxicity.

21. The computer-implemented method of claim 18, wherein indicating the toxicity of the phenotype feature embedding of a sample with compound applied thereto further comprises:
applying the phenotype feature embedding of a sample with compound applied thereto to the second ML model for outputting a lower dimensional embedding of said sample with compound applied thereto; and
applying (324, 325) the lower dimensional embedding of said sample with compound applied thereto to a third ML model trained for outputting an indication of the distance between the lower dimensional embedding and a set of the lower dimensional embeddings associated with the negative control samples.

22. The computer-implemented method of claim 21, further comprising training the third ML model based on performing a grid search over a set of hyperparameters of a high dimensional distance metric algorithm that maximise a distance between the lower dimensional embeddings of the negative control samples and the positive control samples, whilst minimising the distance between the lower dimensional embeddings of the negative control samples or minimising the distance between the lower dimensional embeddings of the positive control samples.

23. The computer-implemented method of claim 16, wherein comparing the distance used for indicating the toxicity of the phenotype feature embedding of a sample is based on Wasserstein distance metrics.

24. The computer-implemented method of claim 16, wherein receiving said set of images associated with the plurality of samples further comprises:
identifying viable samples of cellular structures for analysis in an in-vitro microscopy assay, based on the steps of:
automatically identifying a first set of samples useful for analysis from a plurality of samples of an assay plate;
generating a set of 2-dimensional, 2D, images for each sample in the first set of samples, said set of 2D images for said each sample comprising multiple 2D image slices taken along a z-axis of said each sample; and
identifying from the sets of 2D image slices a set of viable samples; and
outputting data representative of said set of viable samples for analysis as the set of images.

25. The computer-implemented method of claim 24, wherein automatically identifying a first set of samples further comprises, for each sample in the plurality of samples:
pre-processing an image of said each sample;
inputting said pre-processed sample image to a first machine learning, ML, model configured for identifying a region of interest of the input sample image comprising a cellular structure;
inputting the identified region of interest of sample image to a second ML model configured for classifying whether said sample is analysable; and
outputting the first set of samples comprising data representative of those samples that are classified to be analysable.

26. The computer-implemented method of claim 25, wherein:
the first ML model for identifying the region of interest is a convolutional neural network, CNN, or other neural network trained for identifying regions of interest comprising cellular structures, and
the second ML model for classifying whether said sample is analysable is a one class SVM configured to classify whether said region of interest is analysable.
wherein:
training/configuring the CNN based on a labelled training dataset, said labelled training dataset comprising a plurality of images, each of the images annotated with a label comprising data representative of whether a cellular region of interest is present, and/or the location of the region of interest within the image; and
training/configuring the one class SVM to classify whether said region of interest is analysable.

27. The computer-implemented method of claim 24, wherein identifying from the sets of 2D image slices the set of viable samples further comprises, for each sample:
identifying foreground, background and multiple uncertain feature areas of the cellular structure in each of the 2D image slices;
iteratively combining the foreground, background and uncertain feature areas of the 2D image slices to generate a single 2D image of the cellular structure; and
selecting the sample for the viable sample set based on the quality of the single 2D image; and
outputting a set of viable images for each selected sample comprising image data representative of the single 2D images associated with each selected sample;
wherein the multiple uncertain feature areas comprise multiple uncertain foreground features and multiple uncertain background features.

28. An apparatus comprising a processor, a memory unit and a communication interface, wherein the processor is connected to the memory unit and the communication interface, wherein the processor and memory are configured to implement operations for predicting toxicity of one or more compounds applied to a plurality of samples of a cellular structure in an in-vitro microscopy assay, the operations comprising:
receiving a set of images associated with the plurality of samples;
inputting each image of the set of images to a first ML model configured for predicting phenotype features of the cellular structure within the sample associated with said each image;
inputting each of the predicted phenotype features associated with each sample to a second ML model configured for predicting a lower dimensional phenotype feature embedding of said each sample;
comparing the distance between the lower dimensional phenotype feature embedding of said each sample with that of a sample applied with a compound having a known toxicity; and
outputting, for each sample, an indication of the toxicity of said each sample and applied compound thereto based on said comparison.

29. The apparatus of claim 28, wherein the first ML model is a neural network or convolutional neural network, CNN, model, the neural network or CNN model is trained for classification using cellular image training data and the predicted phenotype features are embedded within a full layer of the trained neural network or CNN model, the operations comprising outputting the phenotype feature embeddings from the full layer.

30. The apparatus of claim 28, wherein the second ML model is based on a Uniform Manifold Approximation and Projection, UMAP, algorithm or t-SNE algorithm for dimensional reduction of the phenotype feature embedding of a sample, wherein the phenotype feature embedding is mapped to a lower dimensional vector space for use in comparing the distance between the phenotype feature embedding and that of a sample with a compound having a known toxicity.

31. The apparatus of claim 30, the operations further comprising:
training the second ML model on the UMAP technique using unsupervised training based on negative and positive control samples of the plurality of samples for predicting a toxicity distance metric associated with the samples with compounds applied thereto having a known toxicity; or
training the second ML model comprises iteratively performing a grid search over a set of hyperparameters of the UMAP technique for selecting those hyperparameters that maximise the differences between the negative control samples and the positive control samples.

32. The apparatus of claim 30, wherein indicating the toxicity of the phenotype feature embedding of a sample with compound applied thereto comprises:
applying the phenotype feature embedding of a sample with compound applied thereto to the second ML model for outputting a lower dimensional embedding of said sample with compound applied thereto; and
determining (325, 325a, 325b, 326) an indication of the toxicity of said sample with compound applied thereto based on comparing the distance between said lower dimensional embedding and the embeddings of one or more samples with compounds applied thereto having known toxicity.

33. The apparatus of claim 30, wherein indicating the toxicity of the phenotype feature embedding of a sample with compound applied thereto further comprises:
applying the phenotype feature embedding of a sample with compound applied thereto to the second ML model for outputting a lower dimensional embedding of said sample with compound applied thereto; and
applying (324, 325) the lower dimensional embedding of said sample with compound applied thereto to a third ML model trained for outputting an indication of the distance between the lower dimensional embedding and a set of the lower dimensional embeddings associated with the negative control samples.

34. A non-transitory computer-readable medium comprising data or instruction code, which when executed on a processor, causes the processor to implement operations for predicting toxicity of one or more compounds applied to a plurality of samples of a cellular structure in an in-vitro microscopy assay, the operations comprising:
receiving a set of images associated with the plurality of samples;
inputting each image of the set of images to a first ML model configured for predicting phenotype features of the cellular structure within the sample associated with said each image;
inputting each of the predicted phenotype features associated with each sample to a second ML model configured for predicting a lower dimensional phenotype feature embedding of said each sample;
comparing the distance between the lower dimensional phenotype feature embedding of said each sample with that of a sample applied with a compound having a known toxicity; and
outputting, for each sample, an indication of the toxicity of said each sample and applied compound thereto based on said comparison.

35. The non-transitory computer-readable medium of claim 34, wherein the first ML model is a neural network or convolutional neural network, CNN, model, the neural network or CNN model is trained for classification using cellular image training data and the predicted phenotype features are embedded within a full layer of the trained neural network or CNN model, the operations comprising outputting the phenotype feature embeddings from the full layer.
